SEQUENCE LISTING (26 sequences)

SEQ ID NO: 1 - DNA, 1581 bp, Rous sarcoma virus
  CDS (1)..(1581)
atggggagtagcaagagcaagcctaaggaccccagccagcgccggcgcagcctggagccacccgacagcacccaccacgggggattcccagcctcgcagacccccaacaagacagcagcccccgacacgcaccgcacccccagccgctccttcgggaccgtggccaccgagcccaagctcttcgggggcttcaacacttctgacaccgttacgtcgccgcagcgtgccggggcactggctggcggcgtcaccactttcgtggctctctacgactacgagtcctggattgaaacggacttgtccttcaagaaaggagaacggctgcagattgtcaacaacacggaaggtaactggtggctggctcattccctcactacaggacagacgggctacatccccagtaactatgtcgcgccctcagactccatccaggctgaagagtggtactttgggaagatcactcgtcgggagtccgagcggctgctgctcaaccccgaaaacccccggggaaccttcttggtccgggagagcgagacgacaaaaggtgcctattgcctctccgtttctgactttgacaacgccaaggggctcaatgtgaagcactacaagatccgcaagctggacagcggcggcttctacatcacctcacgcacacagttcagcagcctgcagcagctggtggcctactactccaaacatgctgatggcttgtgccaccgcctgaccaacgtctgccccacgtccaagccccagacccagggactcgccaaggacgcgtgggaaatcccccgggagtcgctgcggctggaggtgaagctggggcagggctgctttggagaggtctggatggggacctggaacggcaccaccagagtggccataaagactctgaagcccggcaccatgtccccggaggccttcctgcaggaagcccaagtgatgaagaagctccggcatgagaagctggttcaactgtacgcagtggtgtcggaagagcccatctacatcgtcattgagtacatgagcaaggggagcctcctggatttcctgaagggagagatgggcaagtacctgcggctgccacagctcgttgatatggctgctcagattgcatccggcatggcctatgtggagaggatgaactacgtgcaccgagacctgcgggcggccaacatcctggtgggggagaacctggtgtgcaaggtggctgactttgggctggcacgcctcatcgaggacaacgagtacacagcacggcaaggtgccaagttccccatcaagtggacagcccccgaggcagccctctatggccggttcaccatcaagtcggatgtctggtccttcggcatcctgctgactgagctgaccaccaagggccgggtgccatacccagggatgggcaacggggaggtgctggaccgggtggagaggggctaccgcatgccctgcccgcccgagtgccccgagtcgctgcatgaccttatgtgccagtgctggcggagggaccctgaggagcggcccactttcgagtacctgcaggcccagctgctccctgcttgtgtgttggaggtcgctgagtag

SEQ ID NO: 2 - protein, 526 aa, Rous sarcoma virus
MGSSKSKPKDPSQRRRSLEPPDSTHHGGFPASQTPNKTAAPDTHRTPSRSFGTVATEPKLFGGFNTSDTVTSPQRAGALAGGVTTFVALYDYESWIETDLSFKKGERLQIVNNTEGNWWLAHSLTTGQTGYIPSNYVAPSDSIQAEEWYFGKITRRESERLLLNPENPRGTFLVRESETTKGAYCLSVSDFDNAKGLNVKHYKIRKLDSGGFYITSRTQFSSLQQLVAYYSKHADGLCHRLTNVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVIEYMSKGSLLDFLKGEMGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMGNGEVLDRVERGYRMPCPPECPESLHDLMCQCWRRDPEERPTFEYLQAQLLPACVLEVAE

SEQ ID NO: 3 - DNA, 1320 bp, Homo sapiens
  CDS (1)..(1320)
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 4 - protein, 439 aa, Homo sapiens
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 5 - DNA, 567 bp, Homo sapiens
  CDS (1)..(567)
atgactgaatataaacttgtggtagttggagctgttggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 6 - protein, 188 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 7 - DNA, 720 bp, Homo sapiens
  CDS (1)..(720)
atggcgcacgctgggagaacggggtacgacaaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgcgacccggtcgccaggacctcgccgctgcagaccccggctgcccccggcgccgccgcggggcctgcgctcagcccggtgccacctgtggtccacctggccctccgccaagccggcgacgacttctcccgccgctaccgcggcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgagccacaagtga

SEQ ID NO: 8 - protein, 239 aa, Homo sapiens [sequence omitted]

SEQ ID NO: 9 - DNA, 3207 bp, Homo sapiens
  CDS (1)..(3207)
atgcctccaagaccatcatcaggtgaactgtggggcatccacttgatgcccccaagaatcctagtagaatgtttactaccaaatgggatgatagtgactttagaatgcctccgtgaggctacgttaataacgataaagcatgaactatttaaagaagcaagaaaataccctctccatcaacttcttcaagatgaatcttcttacattttcgtaagtgttacccaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacgccatggtggctggacaacaaaaatggattggatcttccacacaattaagcagcatgctttgaactga

SEQ ID NO: 10 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDARHGGWTTKMDWIFHTIKQHALN

SEQ ID NO: 11 - DNA, 3207 bp, Bos taurus
  CDS (1)..(3207)
atgcctccaagaccatcatcaggtgaactgtggggcatccacttgatgcccccaagaatcctagtagaatgtttactaccaaatgggatgatagtgactttagaatgcctccgtgaggctacgttaataacgataaagcatgaactatttaaagaagcaagaaaataccctctccatcaacttcttcaagatgaatcttcttacattttcgtaagtgttacccaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaattcgatatggttaaagatccagaagtacaggacttccgaagaaatattctcaatgtttgtaaagaagctgtggatcttagggatcttaattcacctcatagtagagcaatgtatgtttatcctccaaatgtagaatcttcaccagaactgccaaagcacatatataataaattggataaagggcaaataatagtggtgatttgggtaatagtttctccaaataatgacaaacagaagtatactctgaaaatcaaccatgactgtgtgccagaacaagtaattgctgaagcaatcaggaaaaaaactcgaagtatgttgctatcatctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgctgatggctaaagaaagcctctattctcaactgccaatggactgttttacaatgccatcatattccagacgcatctccacagctacgccatatatgaatggagaaacatctacaaaatccctttgggttataaatagtgcactcagaataaaaattctttgtgcaacctatgtgaatgtaaatattcgagacattgacaagatttatgttcgaacaggtatctaccatggaggagaacccttatgtgataatgtgaacactcaaagagtaccttgttccaatcccaggtggaatgaatggctgaattacgatatatacattcctgatcttcctcgtgctgctcgactttgcctttccatttgttctgttaaaggccgaaagggtgctaaagaggaacactgtccattggcctggggaaatataaacttgtttgattacacagatactctagtatctggaaaaatggctttgaatctttggccagtacctcatggactagaagatttgctgaaccctattggtgttactggatcaaatccaaataaagaaactccatgtttagagttggagtttgactggttcagcagtgtggtaaagtttccagatatgtcagtgattgaagagcatgccaattggtctgtatcccgtgaagcaggatttagttattcccatgcaggactgagtaacagactagctagagacaatgaattaagagaaaatgataaagaacagctccgagcaatttgtacacgagatcctctatctgaaatcactgagcaagagaaagattttctgtggagccacagacactattgtgtaactatccccgaaattctacccaaattgcttctgtctgttaaatggaactctagagatgaagtagctcagatgtactgcttggtaaaagattggcctccaatcaagcctgaacaggctatggagcttctggactgcaattacccagatcctatggttcgaggttttgctgttcggtgcttagaaaaatatttaacagatgacaaactttctcagtacctaattcagctagtacaggtactaaaatatgaacagtatttggataacctgcttgtgagatttttactcaaaaaagcgttaactaatcaaaggatcggtcactttttcttttggcatttaaaatctgagatgcacaataaaacagttagtcagaggtttggcctgcttttggagtcctattgccgtgcatgtgggatgtatctgaagcaccttaataggcaagttgaggctatggaaaagctcattaacttgactgacattctcaaacaagagaagaaggatgaaacacaaaaggtacagatgaagtttttagttgagcaaatgcggcgaccagatttcatggatgctctccagggctttctgtctcctctaaaccctgctcatcagctgggaaatctcaggcttgaagagtgtcgaattatgtcttctgcaaaaaggccactgtggttgaattgggagaacccagacatcatgtcagaattactctttcagaacaatgagatcatctttaaaaatggggatgatttacggcaagatatgctaacccttcagattattcgcattatggaaaatatctggcaaaatcaaggtcttgatcttcgaatgttaccttatggatgtctgtcaatcggtgactgtgtgggacttatcgaggtggtgagaaattctcacactataatgcagattcagtgtaaaggaggcctgaaaggtgcactgcagtttaacagccacacactccatcagtggctcaaagacaagaacaagggggaaatatatgatgcggccatcgatttgtttacacgatcatgtgctggatattgtgttgccaccttcattttgggaattggagatcgtcacaatagtaatatcatggttaaagatgatggacaactgtttcatatagattttggacactttttggatcacaagaagaaaaaatttggttataaacgagagcgcgtgccgtttgttttgacacaagatttcttaatagtgattagtaaaggagcccaagaatgcacaaagacaagagaatttgagaggtttcaggagatgtgttacaaggcttatctagctattcggcagcatgccaatctcttcataaatcttttctcaatgatgcttggctctggaatgccagaactgcaatcttttgatgatattgcatacattcgaaagaccctagctttagataaaactgagcaagaggctttggagtatttcatgaaacaaatgaatgatgcacaccatggtggctggacaacaaaaatggattggatcttccacacaattaagcagcatgctttgaactga

SEQ ID NO: 12 - protein, 1068 aa, Bos taurus
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLRAICTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 13 - DNA, 888 bp, Homo sapiens
  CDS (1)..(888)
atggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctga

SEQ ID NO: 14 - protein, 295 aa, Homo sapiens
MEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDNSIRPEELLQMELLLVNKLKWNLAAMTPHDFIEHFLSKMPEAEENKQIIRKHAQTFVALCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACTPTDVRDVDI

SEQ ID NO: 15 - DNA, 1302 bp, Homo sapiens
  CDS (1)..(1302)
atggaggtggtggacccgcagcagctgggcatgttcacggagggcgagctgatgtcggtgggtatggacacgttcatccaccgcatcgactccaccgaggtcatctaccagccgcgccgcaagcgggccaagctcatcggcaagtacctgatgggggacctgctgggggaaggctcttacggcaaggtgaaggaggtgctggactcggagacgctgtgcaggagggccgtcaagatcctcaagaagaagaagttgcgaaggatccccaacggggaggccaacgtgaagaaggaaattcaactactgaggaggttacggcacaaaaatgtcatccagctggtggatgtgttatacaacgaagagaagcagaaaatgtatatggtgatggagtactgcgtgtgtggcatgcaggaaatgctggacagcgtgccggagaagcgtttcccagtgtgccaggcccacgggtacttctgtcagctgattgacggcctggagtacctgcatagccagggcattgtgcacaaggacatcaagccggggaacctgctgctcaccaccggtggcaccctcaaaatctccgccctgggcgtggccgaggcactgcacccgttcgcggcggacgacacctgccggaccagccagggctccccggctttccagccgcccgagattgccaacggcctggacaccttctccggcttcaaggtggacatctggtcggctggggtcaccctctacaacatcaccacgggtctgtaccccttcgaaggggacaacatctacaagttgtttgagaacatcgggaaggggagctacgccatcccgggcgactgtggccccccgctctctgacctgctgaaagggatgcttgagtacgaaccggccaagaggttctccatccggcagatccggcagcacagctggttccggaagaaacatcctccggctgaagcaccagtgcccatcccaccgagcccagacaccaaggaccggtggcgcagcatgactgtggtgccgtacttggaggacctgcacggcgcggacgaggacgaggacctcttcgacatcgaggatgacatcatctacactcaggacttcacggtgcccggacaggtcccagaagaggaggccagtcacaatggacagcgccggggcctccccaaggccgtgtgtatgaacggcacagaggcggcgcagctgagcaccaaatccagggcggagggccgggcccccaaccctgcccgcaaggcctgctccgccagcagcaagatccgccggctgtcggcctgcaagcagcagtga

SEQ ID NO: 16 - protein, 433 aa, Homo sapiens
MEVVDPQQLGMFTEGELMSVGMDTFIHRIDSTEVIYQPRRKRAKLIGKYLMGDLLGEGSYGKVKEVLDSETLCRRAVKILKKKKLRRIPNGEANVKKEIQLLRRLRHKNVIQLVDVLYNEEKQKMYMVMEYCVCGMQEMLDSVPEKRFPVCQAHGYFCQLIDGLEYLHSQGIVHKDIKPGNLLLTTGGTLKISALGVAEALHPFAADDTCRTSQGSPAFQPPEIANGLDTFSGFKVDIWSAGVTLYNITTGLYPFEGDNIYKLFENIGKGSYAIPGDCGPPLSDLLKGMLEYEPAKRFSIRQIRQHSWFRKKHPPAEAPVPIPPSPDTKDRWRSMTVVPYLEDLHGADEDEDLFDIEDDIIYTQDFTVPGQVPEEEASHNGQRRGLPKAVCMNGTEAAQLSTKSRAEGRAPNPARKACSASSKIRRLSACKQQ

SEQ ID NO: 17 - DNA, 3633 bp, Homo sapiens
  CDS (1)..(3633)
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcatgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggcgggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 18 - protein, 1210 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLIMQLMPFGCLLDYVREHKDNIGSQYLLNWCVQIAKGMNYLEDRRLVHRDLAARNVLVKTPQHVKITDFGRAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 19 - DNA, 1761 bp, Homo sapiens
  CDS (1)..(1761)
atgttgtacctggaaaacaatgcccagactcaatttagtgagccacagtacacgaacctggggctcctgaacagcatggaccagcagattcagaacggctcctcgtccaccagtccctataacacagaccacgcgcagaacagcgtcacggcgccctcgccctacgcacagcccagctccaccttcgatgctctctctccatcacccgccatcccctccaacaccgactacccaggcccgcacagtttcgacgtgtccttccagcagtcgagcaccgccaagtcggccacctggacgtattccactgaactgaagaaactctactgccaaattgcaaagacatgccccatccagatcaaggtgatgaccccacctcctcagggagctgttatccgcgccatgcctgtctacaaaaaagctgagcacgtcacggaggtggtgaagcggtgccccaaccatgagctgagccgtgaattcaacgagggacagattgcccctcctagtcatttgattcgagtagaggggaacagccatgcccagtatgtagaagatcccatcacaggaagacagagtgtgctggtaccttatgagccaccccaggttggcactgaattcacgacagtcttgtacaatttcatgtgtaacagcagttgtgttggagggatgaaccgccgtccaattttaatcattgttactctggaaaccagagatgggcaagtcctgggccgacgctgctttgaggcccggatctgtgcttgcccaggaagagacaggaaggcggatgaagatagcatcagaaagcagcaagtttcggacagtacaaagaacggtgatggtacgaagcgcccgtttcgtcagaacacacatggtatccagatgacatccatcaagaaacgaagatccccagatgatgaactgttatacttaccagtgaggggccgtgagacttatgaaatgctgttgaagatcaaagagtccctggaactcatgcagtaccttcctcagcacacaattgaaacgtacaggcaacagcaacagcagcagcaccagcacttacttcagaaacagacctcaatacagtctccatcttcatatggtaacagctccccacctctgaacaaaatgaacagcatgaacaagctgccttctgtgagccagcttatcaaccctcagcagcgcaacgccctcactcctacaaccattcctgatggcatgggagccaacattcccatgatgggcacccacatgccaatggctggagacatgaatggactcagccccacccaggcactccctcccccactctccatgccatccacctcccactgcacacccccacctccgtatcccacagattgcagcattgtcagtttcttagcgaggttgggctgttcatcatgtctggactatttcacgacccaggggctgaccaccatctatcagattgagcattactccatggatgatctggcaagtctgaaaatccctgagcaatttcgacatgcgatctggaagggcatcctggaccaccggcagctccacgaattctcctccccttctcatctcctgcggaccccaagcagtgcctctacagtcagtgtgggctccagtgagacccggggtgagcgtgttattgatgctgtgcgattcaccctccgccagaccatctctttcccaccccgagatgagtggaatgacttcaactttgacatggatgctcgccgcaataagcaacagcgcatcaaagaggagggggagtga

SEQ ID NO: 20 - protein, 586 aa, Homo sapiens
MLYLENNAQTQFSEPQYTNLGLLNSMDQQIQNGSSSTSPYNTDHAQNSVTAPSPYAQPSSTFDALSPSPAIPSNTDYPGPHSFDVSFQQSSTAKSATWTYSTELKKLYCQIAKTCPIQIKVMTPPPQGAVIRAMPVYKKAEHVTEVVKRCPNHELSREFNEGQIAPPSHLIRVEGNSHAQYVEDPITGRQSVLVPYEPPQVGTEFTTVLYNFMCNSSCVGGMNRRPILIIVTLETRDGQVLGRRCFEARICACPGRDRKADEDSIRKQQVSDSTKNGDGTKRPFRQNTHGIQMTSIKKRRSPDDELLYLPVRGRETYEMLLKIKESLELMQYLPQHTIETYRQQQQQQHQHLLQKQTSIQSPSSYGNSSPPLNKMNSMNKLPSVSQLINPQQRNALTPTTIPDGMGANIPMMGTHMPMAGDMNGLSPTQALPPPLSMPSTSHCTPPPPYPTDCSIVSFLARLGCSSCLDYFTTQGLTTIYQIEHYSMDDLASLKIPEQFRHAIWKGILDHRQLHEFSSPSHLLRTPSSASTVSVGSSETRGERVIDAVRFTLRQTISFPPRDEWNDFNFDMDARRNKQQRIKEEGE

SEQ ID NO: 21 - DNA, 3399 bp, Homo sapiens
  CDS (1)..(3399)
atgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgttgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcggggctgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactaa

SEQ ID NO: 22 - protein, 1132 aa, Homo sapiens [sequence omitted]

SEQ ID NO: 23 - DNA, 912 bp, Homo sapiens
  CDS (1)..(912)
atggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagtga

SEQ ID NO: 24 - protein, 303 aa, Homo sapiens
MATSRYEPVAEIGVGAYGTVYKARDPHSGHFVALKSVRVPNGGGGGGGLPISTVREVALLRRLEAFEHPNVVRLMDVCATSRTDREIKVTLVFEHVDQDLRTYLDKAPPPGLPAETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFPPRGPRPVQSVVPEMEESGAQLLLEMLTFNPHKRISAFRALQHSYLHKDEGNPE

SEQ ID NO: 25 - DNA, 312 bp, Homo sapiens
  CDS (1)..(312)
atgtacccatacgatgttccagattacgctccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 26 - protein, 103 aa, Homo sapiens
MYPYDVPDYAPGSTKRALPNNTSSSPQPKKKPLDGEYFTLQIRGRERFEMFRELNEALELKDAQAGKEPGGSRAHSSHLKSKKGQSTSRHKKLMFKTEGPDSD

(19) United States
(12) Patent Application Publication
(10) Pub. No.: US 2013/0145486 A1
(43) Pub. Date: Jun. 6, 2013

(54) METHOD FOR PRODUCING TUMOR CELL

(71) Applicants: Tsuyoshi Akagi, Kobe-shi (JP); Ken Sasai, Kobe-shi (JP)

(72) Inventors: Tsuyoshi Akagi, Kobe-shi (JP); Ken Sasai, Kobe-shi (JP)

(21) Appl. No.: 13/698,186

(22) PCT Filed: May 31, 2011
(86) PCT No.: PCT/JP2011/062876

§ 371 Date: Jan. 17, 2013

(30) Foreign Application Priority Data
May 31, 2010 (JP) 2010-125256

(51) Int. Cl. C12N 15/85

(52) U.S. Cl. CPC C12N 15/85
USPC 800/10; 435/455; 435/325; 435/29

(57) ABSTRACT

The object of the invention is to provide a method for producing tumor cells by carrying out gene transfer into cells derived from normal cells. The invention provides a method for producing tumor cells by transferring cancer-associated genes into immortalized small airway epithelial cells.

### TECHNICAL FIELD

The present invention relates to a method for producing tumor cells by gene transfer based on small airway epithelial cells, tumor cells produced by such a method, and a method for screening antitumor drugs using such tumor cells.

### BACKGROUND ART

Each year, more than one million people die as a result of lung cancer. Indeed, lung cancer can be regarded as the most common type of cancer in both men and women. Non-small cell lung cancer accounts for 80% of all types of lung cancer with associated adenocarcinoma. In most cases, diagnosis takes place at an advanced stage of the disease and, despite the progress that has been made in methods of early detection and treatment, a good prognosis is difficult to come by (Non-Patent Documents 1 and 2). Although molecular analysis of the state and progression of non-small cell lung cancer is expected to lead to improvements in methods of treatment and prevention, at present, effective methods of treatment and prevention have yet to be established. Identifying the minimal and most important changes associated with the tumorigenic transformation of cells is essential to finding the most suitable targets for initial detection and therapeutic intervention. In order to identify such changes, in-vitro cancer model systems obtained by the genetic manipulation of normal human cells are being developed. Because existing cell lines established from tumor tissue contain unknown genetic aberrations, they have not been suitable for research on tumorigenic transformation through genetic change. However, some of in-vitro cancer model systems are reported to be useful in directly elucidating the influence of specific genetic changes on tumorigenic transformation (see Non-Patent Documents 3 and 4).

A number of research groups are conducting studies on the immortalization and oncogenic transformation of human lung epithelial cells (Non-Patent Documents 5 to 7). In these studies, three types of genetic changes observed in most human non-small cell lung cancers, namely, telomerase reverse transcriptase gene introduction and inactivation of the pRB and p53 pathways, are being tested by genetic manipulation using retroviruses. The high expression of telomerase is observed in substantially all lung cancers (Non-Patent Document 8), p53 loss of function is observed in about 50% of non-small cell lung cancers, and an inability to express p16 protein due to promoter region methylation or homozygous deletion is observed in about 70% of non-small cell lung cancer (Non-Patent Document 9).

However, in spite of the advances being made in such research, the literature to date is substantially devoid of reports on the successful artificial production of lung cancer cells which are pathologically very similar to lung cancer tissue isolated from actual lung cancer patients.

At present, cancer cell lines derived from cancer patients are being used in cancer cell research and in the development of cancer drugs. However, because substantially most cancer cell lines derived from patients have unspecified genetic damage, there exists some variability between cell lines in the state of the genetic aberrations. Accordingly, when such cells are used in research on specific molecules or signal pathways, the experimental results obtained cannot be applied to different cell lines. Moreover, because such cells have unspecified genetic damage, they are unsuitable for use in experiments to evaluate the contributions of individual genes to oncogenic transformation.
Non-Patent Document 1: Meuwissen, R., et al., Genes Dev. 19, 643-664 (2005)
Non-Patent Document 2: Herbst, R. S. et al., “Lung cancer,” N. Engl. J. Med. 359, 1367-1380 (2008)
Non-Patent Document 3: Akagi, T., Trends Mol. Med. 10, 542-548 (2004)
Non-Patent Document 4: Zhao, J. J., et al., Trends Mol. Med. 10, 344-350 (2004)
Non-Patent Document 5: Lundberg, A. S., et al., Oncogen 21, 4577-4586 (2002)
Non-Patent Document 6: Ramirez, R. D., et al., Cancer Res. 64, 9027-9034 (2004)
Non-Patent Document 7: Sato, M., et al., Cancer Res. 66, 2116-2128 (2006)
Non-Patent Document 8: Sekido, Y. et al., Annu. Rev. Med. 54, 73-87 (2003)
Non-Patent Document 2: Herbst, R. S. et al., “Lung cancer,” N. Engl. J. Med. 359, 1367-1380 (2008)

### DISCLOSURE OF THE INVENTION

In light of the above, the development of cancer model cells having a defined genetic state has been awaited.

The inventors have successfully achieved the complete tumorigenic transformation of small airway epithelial cells by genetic manipulation. Because the tumorigenic transformed cells thus obtained have been transformed by the genetic manipulation of normal cells, the genetic state has been identified. The inventors, on closely observing these cells, have found that the cells exhibit the histological characteristics of differentiated or undifferentiated human lung cancer cells depending on the combination of genetic elements that have been introduced. In addition, the inventors have succeeded in producing cells having the nature of cancer stem cells for undifferentiated and differentiated human lung cancer.

Accordingly, the invention relates to the following.

[1] A method for producing a tumor cell by transferring a cancer-associated gene(s) into an immortalized small airway epithelial cell, wherein

the immortalized small airway epithelial cell is produced by subjecting a normal small airway epithelial cell to treatments (1) to (3) below:

(1) forced expression of a telomere reverse transcriptase gene;
(2) forced expression of a cyclin-dependent kinase 4 gene; and
(3) induction of p53 loss of function, and wherein

the cancer-associated gene(s) is(are) one or more genes selected from among c-Myc gene, v-Src gene, KRAS mutated gene, BCL2 gene, PIK3 CA mutated gene, Cyclin D1 gene, LKB1 mutated gene, TP63 gene and EGFR mutated gene.

[2] The method according to [1], wherein the cancer-associated gene is a combination of the c-Myc gene and the v-Src gene.
[3] The method according to [1], wherein the cancer-associated gene is a combination of the c-Myc gene, the KRAS mutated gene and the BCL2 gene.
[4] The method according to [2] or [3], wherein the tumor cell is an undifferentiated cancer cell.
[5] The method according to [4], wherein the tumor cell is a cancer stem cell.
[6] The method according to [1], wherein the cancer-associated genes are the c-Myc gene and the v-Src gene, wherein c-Myc gene is inducibly expressed in the immortalized small airway epithelial cell.
[7] The method according to [6], wherein the tumor cell is a poorly differentiated lung cancer cell.
[8] The method according to [1], wherein the cancer-associated gene is a combination of the KRAS mutated gene with one gene selected from among the PIK3CA mutated gene, the Cyclin D1 gene and the LKB1 mutated gene.
[9] The method according to [8], wherein the cancer-associated gene is a combination of the KRAS mutated gene and the Cyclin D1 gene.
[10] The method according to [8], wherein the cancer-associated gene is a combination of the KRAS mutated gene, the Cyclin D1 gene and the TP63 gene.
[11] The method according to [8], wherein the cancer-associated gene is a combination of the KRAS mutated gene and the PIK3 CA mutated gene.
[12] The method according to [8], wherein the cancer-associated gene is a combination of the KRAS mutated gene and the LKB1 mutated gene.
[13] The method according to [1], wherein the cancer-associated gene is a combination of the EGFR mutated gene and the Cyclin D1 gene.
[14] The method according to [8] to [13], wherein the tumor cell is a differentiated lung cancer cell.
[15] The method according to [9], wherein the tumor cell is a cancer stem cell of differentiated lung cancer.
[16] The method according to [8] to [15], wherein the immortalized small airway epithelial cell is a mammalian or primate cell.
[17] The method according to [8] to [15], wherein the immortalized small airway epithelial cell is a human cell.
[18] A tumor cell produced by the method according to any one of [1] to [17].
[19] A tumor-bearing animal model implanted with the tumor cell according to [15].
[20] A method for screening a cancer drug, the method comprising:
(a) contacting a sample derived from the tumor cell of [18] with a candidate substance; and
(b) detecting tumor cell growth inhibiting effects.

Because the tumor cells produced by the method of the invention are cells which have been tumorigenically transformed by genetic manipulation on normal cells, the genetic state thereof has been identified. Therefore, a model system which is highly useful for evaluating the contributions of individual genes in the development of lung cancer is provided by the invention. Of the tumor cells produced by the method of the invention, those cells produced by the transfer of only cancer-associated genes are cells which precisely express the properties of cancer cells, and are thus expected to be highly effective in such applications as the development of cancer drugs.

### BRIEF DESCRIPTION OF THE DRAWINGS

FIG. 1A is a diagram illustrating the tumorigenicity of cells (referred to below as “4T53MS cells”) produced by transferring the c-Myc and v-Src genes into HSAE/4T53 cells, FIG. 1B is a graph showing the tumor growth potential of 4T53MS cells, and FIG. 1C shows images of the results from the hematoxylin-eosin (H&E) staining of 4T53MS cell-derived tumor tissue and the results from immunostaining the tumor tissue.

FIG. 2A is a table showing the results obtained, according to the number of cells implanted, in a study of the tumorigenicity of 4T53MS cells in vivo, and FIG. 2B is a diagram showing tumor formation seven weeks after ten 4T53MS cells were implanted in an NOD-SCID mouse.

FIG. 3A is a diagram showing, in an in-vitro experimental system, that c-Myc expression is induced by doxycycline (DOX) treatment in cells (indicated below as “4T53 mS cells,” wherein ‘m’ is a lower case letter) produced by transferring the inducible c-Myc gene and the v-Src gene into HSAE/4T53 cells, FIG. 3B is a schematic view depicting a method of producing a poorly differentiated lung cancer model. 4T53 mS cells are implanted into nude mice and the mice continue to be administered with doxycycline (DOX). Once tumor formation has been observed, doxycycline administration is stopped. FIG. 3C is a diagram showing the results from immunostaining (cytokeratin staining) 4T53 mS cell-derived tumor tissue.

FIG. 4 is a table showing the tumorigenicity of cell lines established by transferring one or more of various cancer-associated genes (M: c-Myc; R: KRAS^{V12}; B: BCL2; P: PIK3CA^{H1047R}; D: Cyclin-D1; L: LKB1^{D194A}; E: EGFR^{T790M L858R}) into HSAE/4T53 cells.

FIG. 5 presents diagrams showing the results obtained when the indicated genes (M: c-Myc; R: KRAS^{V12}; B: BCL2) were transferred into HSAE/4T53 cells to produce 4T53RM cells (transduced with KRAS^{V12} and c-Myc) and 4T53RMB cells (transduced with KRAS^{V12}, c-Myc and BCL2), the cells thus produced were transplanted into nude mice, which were then subjected to H&E staining (top row), anti-cytokeratin staining (AE1/AE3: middle row), and anti-Vimentin staining (bottom row).

FIG. 6 presents diagrams showing the results obtained from the H&E staining (top row), anti-cytokeratin staining (AE1/AE3: middle row) and anti-Vimentin staining (bottom row) of sections of xenografts derived from 4T53RP cells (left column), 4T53RD cells (center column) and 4T53RL cells (right column) “4T53RP cells” are cells produced by transferring the KRAS^{V12} gene and the PIK3CA mutated gene into HSAE/4T53 cells. “4T53RD cells” are cells produced by transferring the KRAS^{V12} and Cyclin D1 genes into HSAE/4T53 cells. “4T53RL cells” are cells produced by transferring the KRAS^{V12} gene and the LKB1 mutated gene into HSAE/4T53 cells.

FIG. 7 is a schematic diagram showing that various types of lung cancer models can be produced depending on the combination of genes transferred into 4T53 cells.

FIG. 8A shows the results from the anti-p63 staining (left panel), anti-TTF1 staining (center panel) and Alcian Blue staining (right panel) of sections of a xenograft obtained by subcutaneously implanting cell clones from a single 4T53RD cell, and FIG. 8 B is a schematic diagram of the airway epithelium in a mouse.

FIG. 9 is a table showing the results of in vivo tumorigenicity study of cell clones from a single 4T53RD cell, according to the number of cells implanted.

FIG. 10A and FIG. 10B show the results of H&E staining of sections of xenografts derived from 4T53RD cells and 4T53RD Δ Np63 cells respectively.

FIG. 11A, FIG. 11B, and FIG. 11C show the results of H&E staining, Alcian Blue staining, and anti-cytokeratin staining (AE1/AE3) sections of xenografts derived from 4T53ED cells respectively.

### BEST MODE FOR CARRYING OUT THE INVENTION

The invention is described in detail below. The following embodiments are given to illustrate the invention, and are not to be construed as limiting the invention. The invention may be practiced in various forms without departing from the gist thereof.

The entire contents of all reference documents and Japanese Patent Application Laid-open, Japanese Patent Publication and other patent documents cited in this specification are incorporated herein by reference. The entire contents of the specification and diagrams of Japanese Patent Application No. 2010-125256, which was filed on May 31, 2010 and serves as the basis for the priority claim of the present application, are also incorporated herein by reference.

### 1. Method for Producing Tumor Cells

This invention provides a method for producing tumor cells by transferring a cancer-associated gene into an immortalized small airway epithelial cell.

Preferably, the method of the invention is characterized in that the immortalized small airway epithelial cell is produced by subjecting a normal small airway epithelial cell to treatments (1) to (3) below:

(1) forced expression of a telomere reverse transcriptase gene;
(2) forced expression of a cyclin-dependent kinase 4 gene; and
(3) induction of p53 loss of function,
wherein the cancer-associated gene is one or more gene selected from among a c-Myc gene, a v-Src gene, a KRAS mutated gene, a BCL2 gene, a PIK3CA mutated gene, a Cyclin D1 gene, an LKB1 mutated gene, a TP63 gene and an EGFR mutated gene.

### Cancer-Associated Gene

The “cancer-associated gene” used in the method of the invention refers to a gene which, when expressed within a normal cell, has the ability to tumorigenically or oncogenically transform the cell, or to a gene associated with control of the expression of a gene which, when expressed within a normal cell, has the ability to tumorigenically or oncogenically transform the cell. Examples of cancer-associated genes include the myelocytomatosis oncogene (c-Myc), the v-Src gene, the RAS gene, the B-cell leukemia/lymphoma 2 (BCL2) gene, the phosphatidyl inositol 3-kinase catalytic subunit (PIK3CA) gene, the Cyclin D1 gene, the Liver Kinase B1 (LKB1) gene, the c-Abl gene, the c-S is gene, the epidermal growth factor receptor (EGFR) gene, the platelet-derived growth factor receptor (PDGFR) gene, the vascular endothelial growth factor receptor (VEGFR) gene, the HER2/neu gene, the Raf kinase gene, the cyclin-dependent kinase gene, the Tumor protein p63 (TP63) gene, and mutated genes thereof.

In the invention, preferred use may be made of the c-Myc gene, the v-Src gene, the RAS gene, the BCL2 gene, the PIK3CA gene, the Cyclin D1 gene, the LKB1 gene, the EGFR gene, the TP63 gene, and mutated genes and splicing isoforms thereof.

In the invention, the type of cancer-associated gene used is of no particular concern, although a mammalian cancer-associated gene is preferred, a primate cancer-associated gene is more preferred, and a human cancer-associated gene is even more preferred.

The “c-Myr gene” is a gene which encodes a DNA-binding factor associated with regulation of the expression of various genes and DNA replication, i.e., a transcription factor. It has been suggested that an association exists between the impaired expression of the c-Myc gene and various cancers (Dominguez-Sola, D., et al., Nature 448, 445-451 (2007)).

The “v-Src gene” is a cancer-associated gene from the Rous sarcoma virus, which is a type of retrovirus. The sequence for this gene has been described by Mayer B. J., et al., J. Virol. 60 (3), 858-67 (1986).

The “RAS gene” is a gene which encodes a small GTPase that transmits signals which take part in cell differentiation and growth. Cells are known to become cancerous from anomalies in such signal transmission (Goodsell, D. S., Oncologist 4 (3), 263-264 (1999)). RAS gene mutations have been reported in 20 to 30% of non-small cell lung cancers (Aviel-Ronenv, S., et al., Clin. Lung Cancer 1, 30-38 (2006)). In this invention, the RAS gene is preferably a KRAS mutated gene. Here, the KRAS mutated gene is a KRAS gene resulting from the mutation of a wild-type KRAS gene. One example is the gene coding for mutant KRAS^{V12} (SEQ ID NO:6) in which the glycine residue at the 12 position has been replaced with a valine residue. Details on the gene coding for KRAS^{V12} are given in Sato, M. et al, Cancer Res. 66 (4), 2116-2128 (2006).

The “BCL2 gene” is a cancer-associated gene having an apoptosis-suppressing activity, and has been implicated as being associated with melanoma, prostate cancer, breast cancer and lung cancer (Chao, D. T., Korsmeyer, S. J., Annu. Rev. Immunol. 16, 395-419 (1998)).

The “PIK3CA gene” is a gene which encodes a class I PI 3-kinase catalytic subunit, and has been reported to be associated with breast cancer, colorectal cancer and lung cancer (Nature Reviews Cancer 5, 921-929 (2005); Sci. Transl. Med. 2, 26-25 (2010)). In addition, it has been suggested that the PIK3CA gene functions as a cancer-associated gene in the development of uterocervical cancer (Ma, Y. Y., et al., Oncogene 19 (23), 2739-2744 (2000)). In the present invention, the PIK3CA gene is preferably a PIK3CA mutated gene. The PIK3CA mutated gene is a PIK3CA gene that has mutated against its wild type gene, an illustrative example being a gene coding for a PIK3CA^{H1047R} mutated protein (SEQ ID NO:10) in which the histidine residue at the 1047 position has been replaced with arginine.

The “Cyclin D1 gene” is a gene which encodes a cell cycle regulating factor. Overexpression of the Cyclin D1 gene plays a part in the development of parathyroid adenoma, breast cancer, prostate cancer, colorectal cancer, lymphoma, melanoma and lung cancer (Morgan, D. O., Cell 135, 764-794 (2008); Lung Cancer 55, 1-14 (2007)).

The “LKB1 gene” is a gene which codes for Liver Kinase B1, and its overexpression has been implicated in the development of lung cancer, uterocervical cancer, breast cancer, testicular cancer, pancreatic cancer and skin cancer (Katajisto, P., et al., Biochem. Biophys. Acta 1775 (1), 63-75 (2007)). In this invention, the LKB1 gene is preferably a LKB1 mutated gene. The LKB1 mutated gene is a LKB1 gene that has mutations against its wild type gene. For example, preferred use may be made of a gene coding for a dominant negative-type mutant of LKB1 (LKB1-DN). An example of a dominant negative type LKB1 mutant is LKB1^{D194A} (SEQ ID NO:16) in which the aspartic acid at the 194 position has been replaced with alanine.

The “EGFR gene” is a gene coding for an epithelial growth factor receptor. The elevation of the expression of the EGFR gene has been observed in many types of cancer, including breast cancer, colorectal cancer, stomach cancer and brain tumors. EGFR gene mutations are known to be found at a high incidence in adenocarcinoma of the lung (Sharma, S. V., et al., Nat. Rev. Cancer 7 (3), 169-81 (2007)). In the present invention, the EGFR gene is preferably an EGFR mutated gene. The EGFR mutated gene is a EGFR gene that has been mutated against its wild type gene, an illustrative example being the gene coding for the EGFR^{T790M L858R} mutated protein (SEQ ID NO:18) in which the threonine residue at the 790 position has been replaced with a methionine residue and the leucine residue at the 858 position has been replaced with an arginine residue.

The “TP63 gene” is a homolog of the TP53 cancer suppressor gene; cases in which it suppresses cancer and cases in which it promotes cancer are both known. The elevation of the expression of the TP63 gene has been reported in squamous cell cancer of the head and neck and of various organs, including the lungs and esophagus (Deyoung, M. P., et al., Oncogene 26, 5169-5183 (2007)). The TP63 gene in this invention is preferably a gene which encodes the splicing isoform of TP63. An example of a gene which codes for the splicing isoform of TP63 is the TP63^{ΔN} gene which codes for the TP63 splicing isoform which lacks an N-terminal transactivation domain (SEQ ID NO:20).

The respective nucleotide sequences of the human c-Myc, BCL2 and Cyclin D1 genes are available in the NCBI database under the accession numbers indicated below. Details on the respective nucleotide sequences for the KRAS^{V12}, PIK3CA^{H1047R} and LKB1^{D194A} genes can be obtained by referring to the wild-type gene sequences available in the NCBI database under the accession numbers indicated below, as well as to McCoy, M. S. et al., Mol. Cell. Biol. 4, 1577-1582 (1984), Samuels, Y., et al., Science 304, 554 (2004), and Scott, K. D. et al., Cancer Res. 67, 5622-5627 (2007).

TABLE 1

| | | | SEQ | SEQ |
|---|---|---|---|---|
| | NCBI | | ID NO | ID NO |
| Gene name | Accession No. | Species | (gene) | (protein) |
| v-src gene | — | Rous sarcoma virus | 1 | 2 |
| c-Myc gene | V00568 | Homo sapiens | 3 | 4 |
| KRAS gene | NM_004985 | Homo sapiens | — | — |
| KRAS^{V12} gene | — | Homo sapiens | 5 | 6 |
| BCL2 gene | NM_000633 | Homo sapiens | 7 | 8 |
| PIK3CA gene | NM_006218.2 | Homo sapiens | — | — |
| PIK3CA gene | NM_174574.1 | Bos taurus | — | — |
| PIK3CA^{H1047R} gene | — | Homo sapiens | 9 | 10 |
| PIK3CA^{H1047R} gene | — | Bos taurus | 11 | 12 |
| Cyclin D1 gene | NM_053056.2 | Homo sapiens | 13 | 14 |
| LKB1 gene | NM_000455.4 | Homo sapiens | — | — |
| LKB1^{D194A} gene | — | Homo sapiens | 15 | 16 |
| EGFR gene | NM_005228.3 | Homo sapiens | — | — |
| EGFR^{T790M L858R} gene | — | Homo sapiens | 17 | 18 |
| TP63 gene | NM_003722.4 | Homo sapiens | — | — |
| TP63^{ΔN} gene | NM_001114980.1 | Homo sapiens | 19 | 20 |

### Immortalized Small Airway Epithelial Cells

In the invention, the “immortalized small airway epithelial cells” are not subject to any particular limitation, provided they are immortalized cells derived from a small airway epithelial cells. However, immortalized cells from mammalian small airway epithelial cells are preferred, immortalized cells from primate small airway epithelial cells are more preferred, and immortalized cells from human small airway epithelial cells are even more preferred. Of such immortalized cells from small airway epithelial cells, immortalized cells from normal small airway epithelial cells are still more preferred. Here, “normal” refers to a healthy state; that is, a state free of any detectable disease or anomaly.

### Immortalized Human Small Airway Epithelial Cells

In the invention, “immortalized human small airway epithelial cells” are not subject to any particular limitation, provided they are immortalized cells from a human small airway epithelial cells. However, immortalized cells from normal human small airway epithelial cells are preferred. “Normal human small airway epithelial cells” refer to human small airway epithelial cells from a healthy person, such as small airway epithelial cells of a person in a state free of any detectable disease or anomaly.

The above-described immortalized small airway epithelial cells or immortalized human small airway epithelial cells can be produced by subjecting normal small airway epithelial cells or normal human small airway epithelial cells to any one or combination of treatments (1) to (3) below, although carrying out all of treatments (1) to (3) is preferred. The order in which treatments (1) to (3) are carried out is of no particular concern:

(1) forced expression of a telomerase gene;
(2) forced expression of a cyclin-dependent kinase gene;
(3) induction of loss of function of an apoptosis-inducing protein.

Concerning Treatment (1), telomerase is an enzyme which extends specific repeat sequences at the ends of eukaryotic chromosomes. Telomerase is composed of subunits such as telomere reverse transcriptase, telomere RNA component (TERC), dyskerin and telomerase-associated protein 1 (TEP1), each of which is encoded at a different gene loci. The telomerase gene which is forcibly expressed in Treatment (1) is exemplified by the various genes coding for telomere reverse transcriptase, TERC, dyskerin and TEP1. However, in this invention, preferred use can be made of the telomerase reverse transcriptase gene (referred to below as the “TERT gene”) in particular.

Concerning Treatment (2), cyclin-dependent kinases (CDK) take part in turnover of the cell cycle. This family of kinases is composed of CDK1 to 13. The cyclin-dependent kinase (Cdk) gene which is forcibly expressed in Treatment (2) may be a gene coding for any one of CDK1 to 13, although preferred use may be made of the gene coding for CDK4 (the Cdk4 gene). CDK4 is the binding partner of Cyclin D1, and mutations of the Cdk4 gene have been detected in various types of tumors (Zuo, L., et al., Nature Genet. 12, 97-99 (1996)).

Concerning Treatment (3), examples of apoptosis-inducing proteins include p53, caspase-3, caspase-8 to caspase-10, and caspase-12. The protein whose loss of function is induced in Treatment (3) may be any of the proteins from among p53, caspase-3, caspase-8 to caspase-10 and caspase-12, although p53 is preferred in this invention. Examples of methods for inducing p53 loss of function include adding a p53 protein-neutralizing antibody, transferring a gene coding for this antibody into the cell, knockout of the TP53 gene, which is the gene coding for p53, knockdown by RNA interference, and forced expression of a dominant negative-type TP53 gene.

An example of a published study on knockdown of the TP53 gene by RNA interference is that by Sato, M., et al. in Cancer Res. 66, 2116-2128 (2006)). Sato, M., et al. have immortalized human airway epithelial cells by carrying out knockdown of the TP53 gene by RNA interference.

In above Treatments (1) to (3), the species of the genes that are forcibly expressed and the species of the protein whose loss of function is induced are of no particular concern, although mammalian genes or proteins are preferred, primate genes or proteins are more preferred, and human genes or proteins are even more preferred.

In the invention, immortalized small airway epithelial cells that are especially preferable for use are cells in which immortality has been acquired by forcibly expressing the TERT gene and the Cdk4 gene and inducing p53 loss of function in normal small airway epithelial cells (referred to below as “SAE cells”). Even more preferred examples are cells in which immortality has been acquired by transferring the TERT gene, the Cd14 gene and the dominant negative-type TP53 gene to a SAE cell, and forcibly expressing these genes.

Immortalized human airway epithelial cells that may be more preferably used are cells in which immortality has been acquired by forced expressed of the hTERT gene and the Cdk4 gene in normal human small airway epithelial cells (referred to below as “HSAE cells”), and by the induction of p53 loss of function. Even more preferred examples are cells (referred to below as “HSAE/4T53 cells”) in which immortality has been acquired by transferring the hTERT gene, the Cdk4 gene and the dominant negative-type TP53 gene to a HSAE cell, and forcibly expressing these genes. The nucleotide sequences of the hTERT gene, the Cdk4 gene and the TP53 gene are available in the NCBI database under the following accession numbers. For the nucleotide sequences of the dominant negative-type TP53 gene, reference may be made to Shaulian, E., et al., Mol. Cell. Biol. 12, 5581 (1992).

TABLE 2

| | | | SEQ | SEQ |
|---|---|---|---|---|
| | NCBI | | ID NO | ID NO |
| Gene name | Accession No. | Species | (gene) | (protein) |
| hTERT gene | NM_198253.2 | Homo sapiens | 21 | 22 |
| Cdk4 gene | NM_000075.2 | Homo sapiens | 23 | 24 |
| TP53 gene | NM_000546.4 | Homo sapiens | — | — |
| Dominant | — | Homo sapiens | 25 | 26 |
| negative-type | | | | |
| TP53 gene | | | | |

The SAE cells used may be ones collected from a mammal Alternatively, cells that are commercially available (from CELLnTEC, in Bern, Switzerland) may be used.

The HSAE cells used may be ones collected from a human. Alternatively, cells that are commercially available (from Lonza, in Walkersville, Md., USA) may be used.

The method of transferring genes to normal small airway epithelial cells or to normal human small airway epithelial cells is similar to the method of transferring genes to immortalized human small airway epithelial cells that is described below.

### Gene Transfer Method

In the invention, when transferring a cancer-associated gene into an immortalized human small airway epithelial cell, typically the cancer-associated gene is inserted into an expression vector as a suitable expression cassette, and the immortalized human small airway epithelial cell is transformed with the vector. A suitable expression cassette includes at least (i) to (iii) below as constituent elements:

(i) a promoter which can be transcribed in an immortalized human small airway epithelial cell;
(ii) a cancer-associated gene bound to the promoter; and
(iii) a sequence which encodes an RNA molecule transcription termination and polyadenylation signal.

Examples of promoters which can transcribed in immortalized human small airway epithelial cells include, but are not limited to, CMV, CAG, LTR, EF-1a and SV40 promoters.

The expression vector may have, other than the above expression cassette, a selective marker expression cassette for selection of the transformed immortalized human small airway epithelial cell. Examples of selective markers include, but are not limited to, positive selection markers such as neomycin-resistant genes and the hygromycin B phosphotransferase gene; expression reporters such as the LacZ, GFP (Green Fluorescence Protein) and luciferase genes; and negative selection markers such as the herpes simplex virus thymidine kinase gene (HSV-TK) and the diphtheria toxin A fragment (DTA).

Transformed immortalized human small airway epithelial cells that have been transformed can easily be selected by means of the above markers. For example, in the case of cells to which a neomycin-resistant gene has been transferred as a marker, primary selection can be carried out by culturing the cells in a medium to which G418 has been added. Alternatively, in cases where the targeting vector includes a gene for a fluorescent protein such as GFP as the marker, in addition to selection by drug resistance, sorting of the fluorescent protein expressing cells can be carried out using a fluorescence activated cell sorter (FACS).

Expression vectors which may be used for transferring a cancer-associated gene into immortalized human small airway epithelial cells are exemplified by known expression vectors capable of gene transfer into cells, including expression vectors of this type that are commercially available. Examples of such expression vectors include pEGFP-C1™ (Clontech), pCMV-HA™ (Clontech), pMSCVpuro™ (Clontech), pEF-DEST51™ (Invitrogen), pCEP4™ (Invitrogen), and ViraPower II Lentiviral Gateway System™ (Invitrogen). The expression vector may be transferred into immortalized human small airway epithelial cells by a known gene transfer method such as electroporation, microinjection, the calcium phosphate method, lipofection or virus infection. For further details on gene transfer methods, reference may be made to Sambrook & Russell, Molecular Cloning: A Laboratory Manual, Vol. 3 (Cold Spring Harbor, Laboratory Press, 2001).

### 2. Tumor Cells

In the invention, “tumor cells” refer to cells which hyperproliferate autonomously in vivo. Examples of tumor cells include cells included in (1) sarcomas such as osteosarcoma and soft tissue sarcoma, (2) carcinomas such as carcinoma of the breast, carcinoma of the lung, carcinoma of the bladder, carcinoma of the thyroid gland, carcinoma of the prostate, carcinoma of the colon, colorectal carcinoma, carcinoma of the pancreas, carcinoma of the stomach, carcinoma of the liver, carcinoma of the uterus, carcinoma of the cervix and carcinoma of the ovary, (3) lymphomas such as Hodgkin lymphoma and non-Hodgkin lymphoma, (4) neuroblastomas, (5) melanomas, (6) myelomas, (7) Wilms tumors, (8) leukemias such as acute myelocytic leukemia (AML), chronic myelocytic leukemia (CML), acute lymphocytic leukemia (ALL) and chronic lymphocytic leukemia (CLL), (9) gliomas, and (10) retinoblastomas.

The “tumor cells” may also be exemplified by, in terms of the degree of differentiation, differentiated cancer cells and undifferentiated cancer cells. In some embodiments of the invention, preferred forms of the tumor cell include undifferentiated cancer cells and differentiated lung cancer cells. Differentiated lung cancer cells may be further classified into poorly differentiated lung cancer cells and differentiated lung cancer cells. Example of poorly differentiated lung cancer cells include large cell cancer-like cells. Examples of differentiated lung cancer cells include squamous cancer cells and adenocarcinoma cells.

Confirmation of the tumorigenic transformation of cells may be carried out by transferring a cancer-associated gene into immortalized human small airway epithelial cells or immortalized human small airway epithelial cells, culturing these cells for several days, then subcutaneously injecting the cultured cells into a suitable model animal and observing tumor mass formation.

The model animal is preferably a mammal other than human, and more preferably an immunosuppressed mammal Examples of immunosuppressed mammals include, but are not limited to, nude rats, nude mice and SCID mice.

Another exemplary method of confirming the tumorigenic transformation of cells involves culturing on soft agar immortalized human small airway epithelial cells into which a cancer-associated gene has been transferred, and observing colony formation by the cells (soft agar colony forming assay). Specifically, this is a method in which immortalized human small airway epithelial cells are prepared with a certain cell concentration, seeded onto a soft agar medium, and the rate of cell proliferation is observed. For details on soft agar colony forming assays, reference may be made to Tanaka, S., et al., Proc. Natl. Acad. Sci. USA 94, 2356-2361 (1997).

### Types of Tumor Cells

In the method of the invention, undifferentiated cancer cells, poorly differentiated lung cancer cells or differentiated lung cancer cells may be selectively produced depending on the combination of cancer-associated genes that are transferred.

### Undifferentiated Cancer Cells:

In the method of the invention, undifferentiated cancer cells can be produced by transferring the combination of genes shown in (A) or (B) below to immortalized human small airway epithelial cells:

(A) c-Myc gene and v-Src gene;
(B) c-Myc gene, KRAS mutated gene and BCL2 gene.

In this method, an N-Myc gene or an L-Myc gene may be used in place of the c-Myc gene, an HRAS gene or an NRAS gene may be used in place of the KRAS gene, or a BCL-X gene may be used in place of the BCL2 gene.

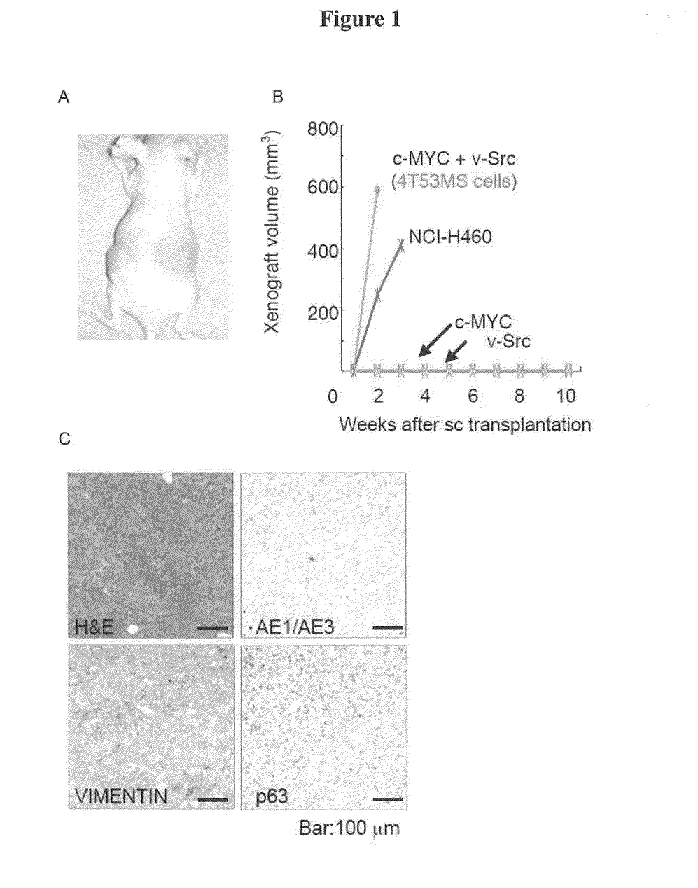

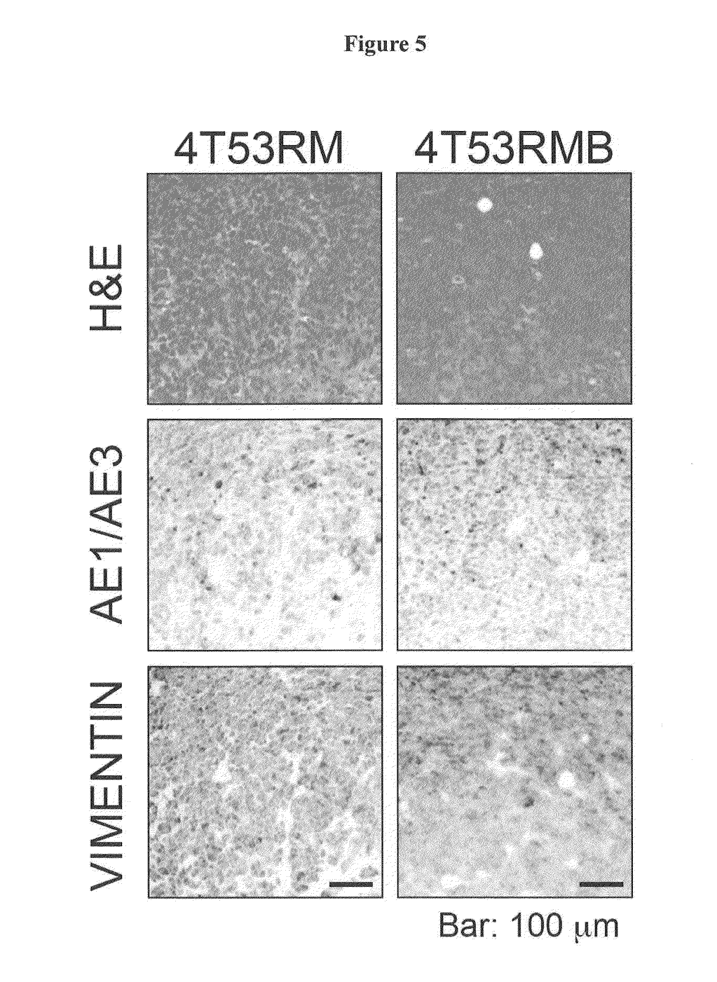

With regard to the undifferentiated cancer cells produced by the above combinations of genes (which cells are referred to below as the “undifferentiated cancer cells of the invention”), in immunohistochemical analysis, most of the cells are negative or weakly positive for the epithelial cell marker cytokeratin (antibody clone name, AE1/AE3), and positive for the mesenchymal marker VIMENTIN (FIGS. 1C and 5). However, the presence of some cells which are positive for AE1/AE3 and negative for VIMENTIN is acceptable (FIG. 1C).

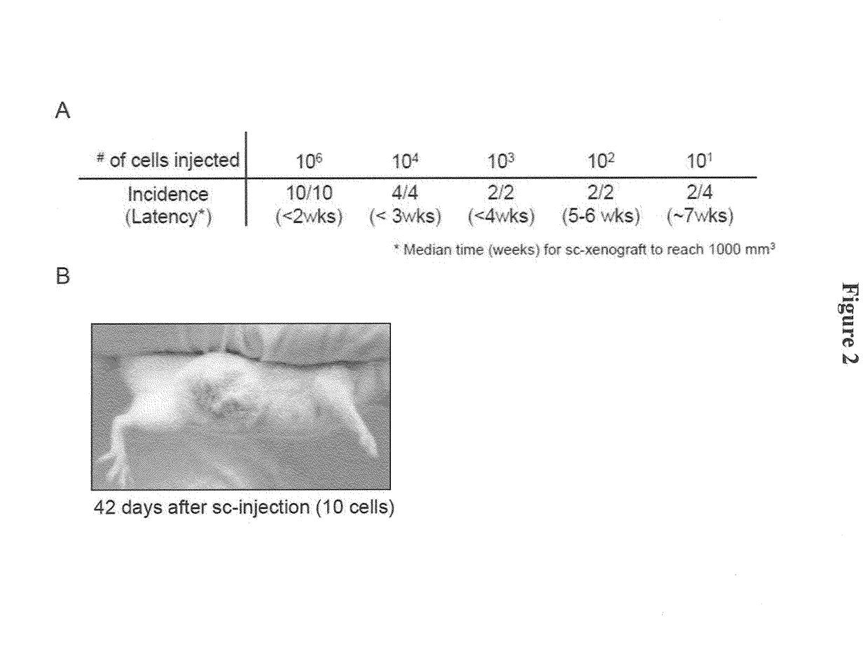

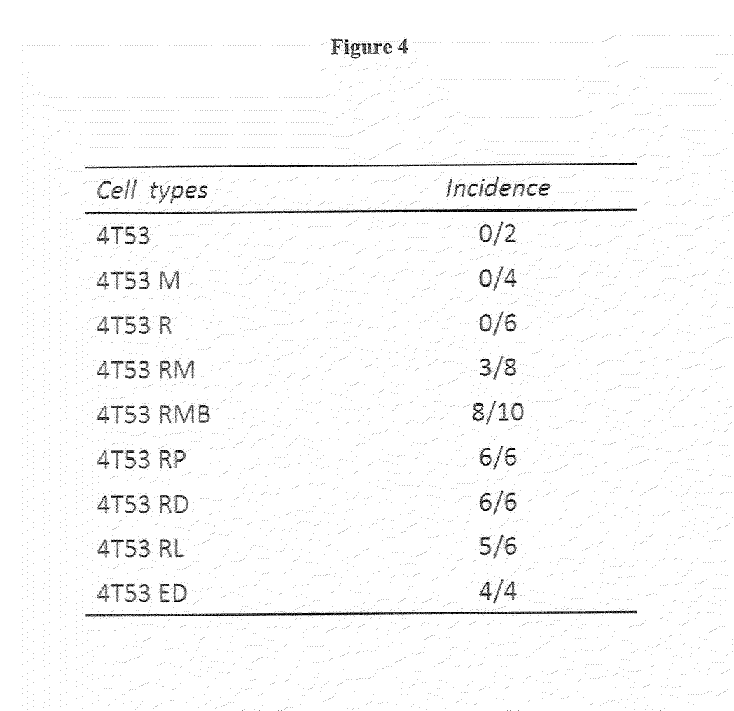

The undifferentiated cancer cells of the invention are characterized with a high tumorigenicity (FIGS. 2 and 4).

Undifferentiated cancer cells produced by the combination of genes in (A) above have, as shown in FIG. 1B, a tumorigenicity about twice as large that of NCI-H460 human non-small cell lung cancer cells (ATCC HTB-177), and are capable of forming tumor tissue even when only ten of the cells have been inoculated subcutaneously in immunodeficient mice (FIG. 2A). As a result, undifferentiated cancer cells produced by the combination of genes in (A) according to the invention have a very strong tumor-initiating ability, which can be regarded as one characteristic of a cancer stem cell.

Undifferentiated cancer cells produced by the combination of genes in (B) above are able to form tumors in nude mice at a high incidence of 80%, and thus have a high tumor-forming ability (FIG. 4).

### Poorly Differentiated Lung Cancer Cells:

In the method of the invention, by transferring the combination of genes shown below in (C) to immortalized human small airway epithelial cells and inductively expressing the c-Myc gene, poorly differentiated lung cancer cells can be produced:

(C) Inducible c-Myc Gene and v-Src Gene.

Here, “inducible c-Myc gene” refers to a c-Myc gene which is capable of inductive expression under an external stimulus, or to an expression system thereof “Inducible c-Myc genes” are encompassed by the above-mentioned “c-Myc genes.” Also, “inductive expression” refers to the control of gene expression by an expression-inducing system. Inductive expression should be started after transfer of the genes indicated in (C) to the immortalized human small airway epithelial cells, and is preferably carried out in the period following transfer to the immortalized human small airway epithelial cells and up to confirmation of tumor formation.

The expression-inducing system is not subject to any particular limitation, provided expression of the target gene (c-Myc gene) can be artificially controlled. Illustrative examples include an expression-inducing system which uses dexamethasone and a mouse mammary tumor virus (MMTV) promoter, an expression-inducing system (Tet-on system) which uses tetracycline, doxycycline or the like and a tetracycline-responsive promoter, an expression-inducing system which uses metal ions such as nickel, cobalt, manganese or iron and a metallothionein promoter, and an expression-inducing system which uses a heat shock protein promoter.

Alternatively, once the c-Myc gene has been forcibly induced and tumor formation has been confirmed, the c-Myc function may be destroyed using a system which suppresses expression of the c-Myc gene (e.g., a Tet-off system) or a system which degrades the c-Myc protein (Auxin-based degron system: Nishimura, K. et al., Nature Methods, Vol. 6, No. 12 (December 2009)). Advantageous use can be made of a Tet-on system, with the addition of doxycycline being preferred from the standpoint of sensitivity.

Also, in the present invention, an N-Myc gene or an L-Myc gene may be used instead of the c-Myc gene.

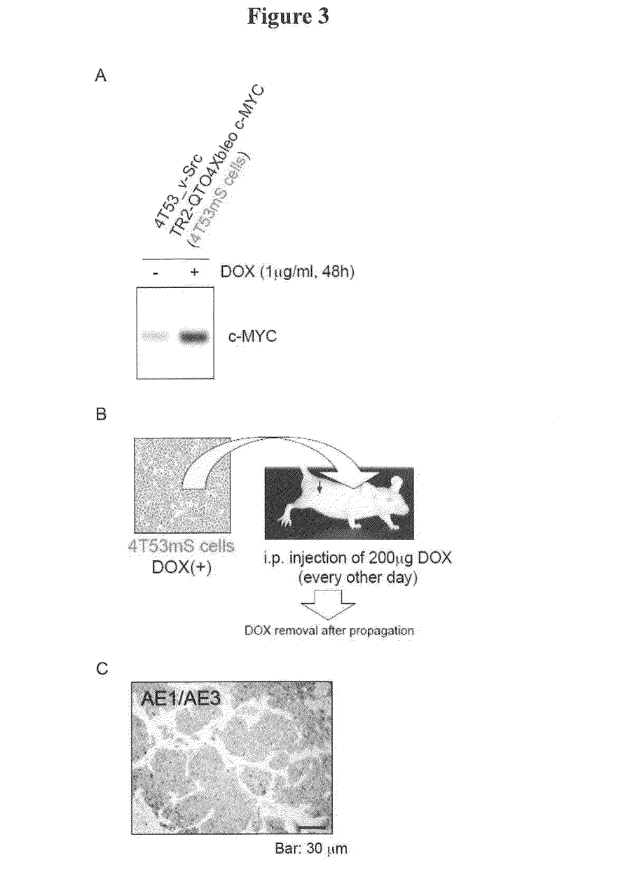

The poorly differentiated lung cancer cells produced with the above combinations of genes (which cells are referred to below as “poorly differentiated lung cancer cells of the invention”) exhibit the large cell cancer-like morphological feature of being positive for the epithelial cell marker cytokeratin (antibody clone name, AE1/AE3) (FIG. 3C).

### Differentiated Lung Cancer Cells:

In the method of the invention, differentiated lung cancer cells can be produced by transferring the combination of genes shown in any of (D) to (H) below to immortalized human small airway epithelial cells:

(D) KRAS mutated gene and PIK3CA mutated gene;
(E) KRAS mutated gene and Cyclin D1 gene;
(F) KRAS mutated gene and LKB1 mutated gene;
(G) KRAS mutated gene, Cyclin D1 gene and TP63 gene;
(H) Cyclin D1 gene and EGFR mutated gene.

In the invention, an HRAS gene or an NRAS gene may be used instead of the KRAS gene, or a Cyclin D2 gene or a Cyclin D3 gene may be used instead of the Cyclin D1 gene.

Differentiated lung cancer cells produced with the above gene combinations (which cells are referred to below as “differential lung cancer cells of the invention”) are all characterized by having a very high tumorigenicity (FIG. 4).

Of the differentiated lung cancer cells of the invention, those cells produced with combination (D) or (H) exhibit a morphology in which squamous epithelial-like cells and adenocarcinoma-like cells are intermixed, whereas those cells produce with combination (E) or (F) each have the morphology of adenocarcinoma-like cells. Cells produced with combination (G) have the morphology of squamous epithelial-like cells.

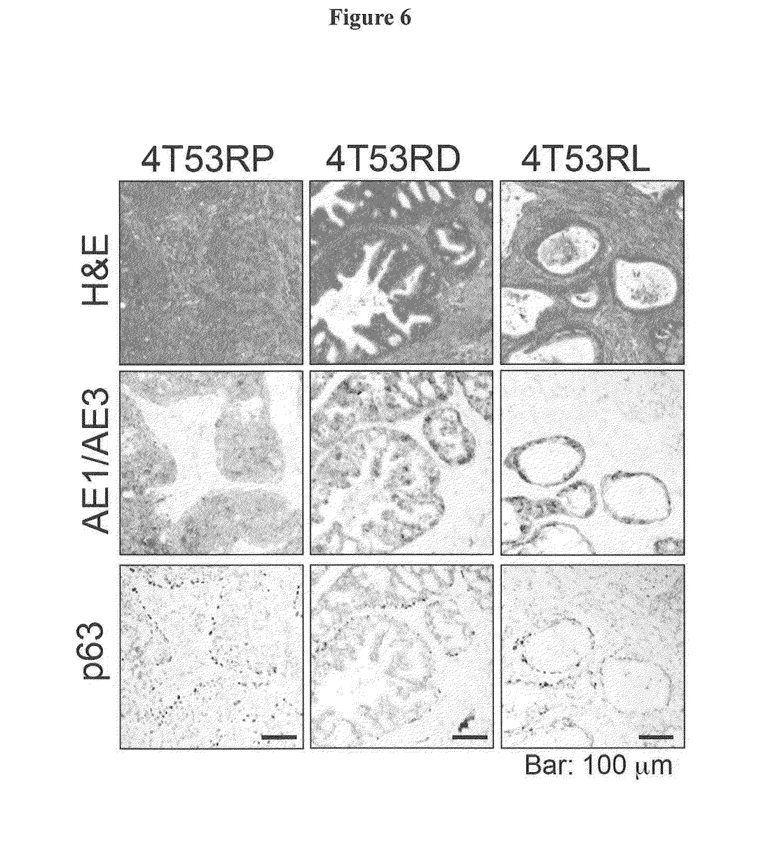

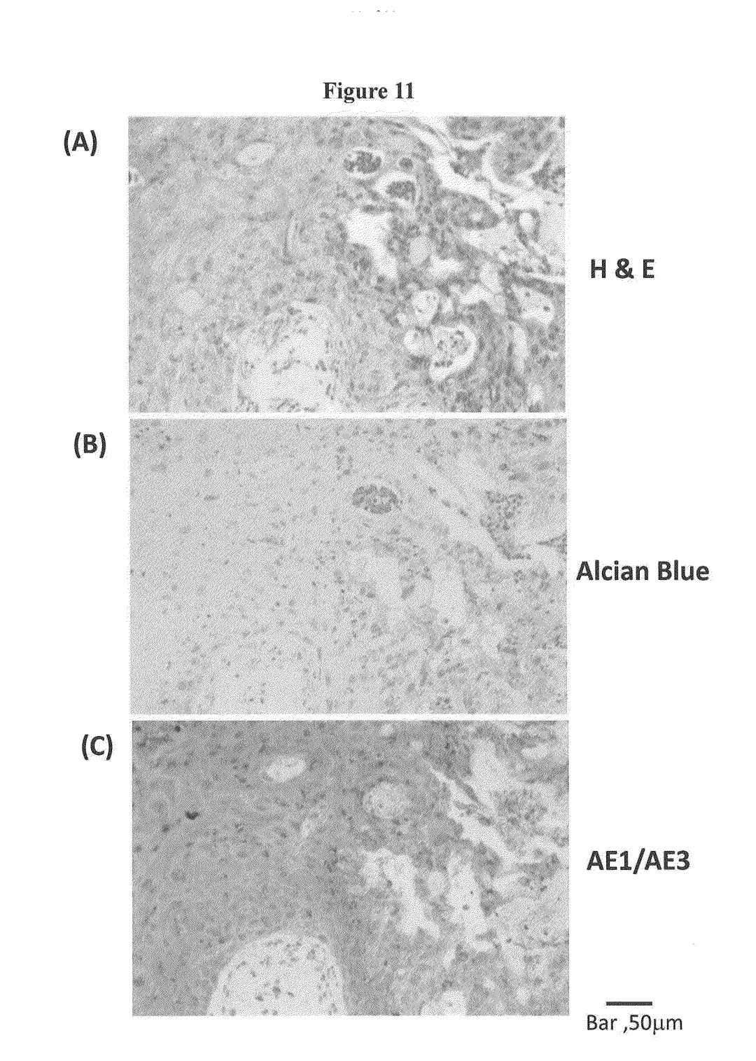

In immunohistochemical analysis, differentiated lung cancer cells produced with any one of combinations (D), (E) and (F) are positive for AE1/AE3 and p63 (FIG. 6). Differentiated lung cancer cells produced with combination (H) are positive for AE1/AE3 (FIG. 11C) and positive for Alcian Blue staining (FIG. 11B).

The tumor cells of the invention all have a high tumor incidence and ability to proliferate. When these cells are implanted in model animals and induced to form tumors, the tumors thus formed show very similar pathology to tumor tissue which forms in the animal species to which the small airway epithelial cells prior to transfer of the cancer-associated gene belong. For example, when a human small airway epithelial cell is used, thus formed tumor shows very similar pathology to that of a cancer tissue, especially lung cancer tissue, isolated from human cancer patients. Therefore, the tumor cells of the invention are very useful in a wide variety of lung cancer research, such as research on the reprogramming, differentiation and biological mechanisms of lung cancer cells, the identification of target molecules for lung cancer treatment, and the screening of cancer drugs.

### 3. Tumor-Bearing Animal Model

In the invention, “tumor-bearing animal model” refers to a non-human animal model in which the above-described tumor cells have been implanted and a tumor mass has been formed.

The model animal is preferably a mammal other than human, examples of which include, but are not limited to, mouse, rat, pig, dog, monkey, hamster and rabbit. Of these model animals, an immunosuppressed mammal is especially preferred. Immunosuppressed mammals can be created by administering an immunosuppressant such as cyclosporine to an ordinary mammal, although a mammal in which immunity has been congenitally suppressed on account of the genetic background is preferred. Examples of congenitally immunosuppressed mammals include, but are not limited to, nude rats, nude mice and SCID mice.

No particular limit is imposed on the method of transferring the above-described tumor cells to the model animal. A method that is conventionally used in the art may be adequately selected according to the model animal in which implantation is to be carried out. As an instance of implantation in an animal other than mice, reference may be made to, for example, “Genetic induction of tumorigenesis in swine,” Oncogen 26, 1038-1045 (Sep. 11, 2006). Also, insofar as the tumor cells used at the time of implantation form tumor masses when implanted in the model animals, the model animals may be of the same species or of different species.

From the standpoint of the ease of re-extracting the implanted tumor cells, implantation by subcutaneous injection or intraperitoneal injection is preferred. On the other hand, from the anatomical standpoint, orthotopic implantation is preferred.

### 4. Method of Screening for Cancer Drugs

The present invention provides a method for screening cancer drugs, which method includes the steps of:

(a) contacting a sample derived from the tumor cells of the invention with a candidate substance; and

(b) detecting tumor cell growth inhibiting effects.

“A sample derived from the tumor cells” refers to tumor cells or tumor tissue, or to a sample that has been suitably prepared so as to be easy to use in the screening method steps.

“Contacting a sample derived from the tumor cells with a candidate substance” means that the candidate substance approaches to a degree such as to give rise to interactions with molecules at the surface of the tumor cells, binds with such molecules, or is taken up into the tumor cells. In cases where the tumor cells are cultured cells, the cells and the candidate substance can be brought into contact by adding the candidate substance to at least a given concentration to the culture medium in contact with the cells. On the other hand, in cases where the tumor cells have been implanted into the body of an animal, the tumor cells and the candidate substance can be brought into contact by administering a given amount of the candidate substance to the animal. In such a case, the route of administration is not particularly limited, provided it is a route commonly used for administering the candidate substance. Illustrative examples of suitable routes of administration include oral, sublingual, nasal, pulmonary, gastrointestinal and percutaneous administration, ocular instillation, intravenous injection, subcutaneous injection, intramuscular injection, intraperitoneal injection, localized injection, and surgical implantation. Preferred routes are oral administration, intraperitoneal injection and intravenous injection.

Exemplary candidate substances include drugs which have already been confirmed to have antitumor effects, and compounds, polypeptides, nucleic acids, antibodies and low-molecular-weight compounds having latent antitumor effects. Illustrative examples of such candidate substances include, but are not limited to, antimetabolites (e.g., 5-fluorourasyl (5-FU)), antagonists of folic acid metabolism (e.g., the dihydropteroic acid synthase inhibitors sulfadiazine and sulfamethoxazole, and the dihydrofolic acid reductase inhibitors (DHFR inhibitors) methotrexate, trimethoprim and pyrimethamine), inhibitors of pyrimidine metabolism (e.g., the thymidylic acid synthase inhibitors 5-FU and flucytosine (5-FC)), inhibitors of purine metabolism (e.g., the IMPDH inhibitors 6-mercaptopurine and the prodrug thereof azathioprine), adenosine deaminase (ADA) inhibitors (e.g., pentostatin), ribonucleotide reductase inhibitors (the ribonucleotide reductase inhibitor hydroxyurea), nucleotide analogs (the purine analogs thioguanine, fludarabine phosphate and cladribine; the pyrimidine analogs cytarabine and gemcitabine), L-asparaginase, alkylating agents (e.g., the nitrogen mustards cyclophosphamide, melphalan and thiotepa; the platinum agents cisplatin, carboplatin and oxaliplatin; and the nitrosoureas dacarbazine, procarbazine and ranimustine), antitumor antibiotics (sarkomycin, mitomycin C, doxorubicin, epirubicin, daunorubicin), topoisomerase inhibitors (irinotecan, nogitecan, doxorubicin, etoposide, levofloxacin, ciprofloxacin), microtubule inhibitors (vinblastin, Vincristine, vindesine), colhicine, microtubule inhibitors (paclitaxel, docetaxel), molecularly targeted drugs (trastuzumab, rituximab, imatinib, gefitinib, bortezomib, erlotinib), steroids (e.g., dexamethasone), finasteride, aromatase inhibitors, tamoxifen, and combinations thereof.

To determine the tumor cell growth inhibiting effects, two systems may be prepared, either in the form of a culture system wherein the same number of cells are seeded in culture tissue at the same cell concentration and cultured under the same conditions, or in the form of a transplantation animal model of the same line wherein the same number of cells are transplanted in the same cell concentration. The candidate substance is brought into contact with the cells of one system (the sample), but is not brought into contact with the cells of the other system (the control), and tumor cell growth in both systems is observed. The growth inhibiting effects can be ascertained by measuring and comparing the numbers of tumor cells present in the two systems after a given period of time has elapsed.

In cases where the tumor cells of the invention are used for screening in the form of cultured cells, after the candidate substance has been brought into contact with the sample cells, the number of sample cells and the number of control cells are measured with a cell counter or the like, and the tumor growth effects can be determined by comparing the respective numbers of cells.

In cases where the tumor cells of the invention are used for screening by implantation in an animal model, after the candidate substance has been administered to the animal model serving as the sample system, tumor tissue is removed from both sample animals and control animals, and the numbers of cells present in tumor tissue from the sample animals and from the control animals can be measured and compared. Alternatively, the tumor growth effects can be determined by removing the tumor tissue and comparing the volume of the tumor tissue from the sample animals and the control animals. The tumor volume can be determined using the following formula.

Tumor volume=ab²/2 (where a is the width, and b is the length)

It is also possible, after administering the candidate substance to both sample and control animals, to determine the tumor growth effect by comparing the incidence of measurable tumor formation at the tumor cell implantation site in the sample animals and the control animals.

The animal model is the same as the animal model described above under “2. Tumor Cells.” No particular limitation is imposed on the method of implantation in the animal model, although subcutaneous injection or intraperitoneal injection is preferred on account of the ease of re-extracting the implanted tumor cells.

In cases where the rate of increase in tumor cells within a sample system is smaller than the rate of increase in tumor cells within a control system, it may be concluded that the candidate substance has an antitumor effect. Alternatively, in cases where ample data on the rate of increase under given conditions already exists for the tumor cell of the invention, such determinations may be made by comparison with a baseline derived from average values, standard deviations, etc. obtained by the statistical treatment of such data.

Average values, standard deviations, etc. for the tumor rate of increase can be obtained by various statistical methods. Specifically, in the case of cultured cells, by using as the parameters the initial number of cells and the cell density at the time of seeding, or, in the case of implanted cells, by using as the parameters the weight of the animal model at the time of implantation and the number of tumor cells implanted, these values can be determined by two-way ANOVA analysis using statistical analysis software such as IBM SSPS Statistics 18 (SSPS). It is possible to further enhance the accuracy of analysis by adding the rate of increase in tumor cells obtained from carrying out the method of the invention as new data to a population for statistical analysis and thus increasing the parameters.

Because the tumor cells of the invention exhibit characteristics which are pathologically very similar to lung cancer cells isolated from patients, it is expected that cancer drugs confirmed by the screening method of the invention to have tumor cell growth-suppressing effects will also have antitumor effects when used in the treatment of actual cancer patients, particularly lung cancer patients.

The invention is illustrated more fully below by way of examples, although the invention is not limited to the modes thereof described in the examples.

### EXAMPLES

The procedures of the experiments carried out in the examples are described below.

### Retrovirus Vector and Retrovirus-Mediated Gene Transfer

Of the genes shown in Table 1, v-Src (SEQ ID NO:1) and KRAS^{V12} (SEQ ID NO:5) were inserted into the pCX4pur vector (GenBank#: AB086386). c-Myc (SEQ ID NO:3), PIK3CA^{H1047R} (SEQ ID NO:11), Cyclin D1 (SEQ ID NO:13) and LKB1^{D194A} (SEQ ID NO:15) were inserted into the pCX4bleo vector (GenBank#: AB086388). BCL2 (SEQ ID NO:7) was inserted into the pCX4redEx vector (GenBank#: AB296084). The EGFR^{T79M L858R} gene (SEQ ID NO:17) was inserted into the pCX4pur vector (GenBank Accession No. AB086386). The TP63^{ΔN} gene (SEQ ID NO:19) was inserted into the pCX4hyg vector (GenBank Accession No. AB086387). Of the genes shown in Table 2, hTERT (SEQ ID NO:21) was inserted into the pCX4neo vector (GenBank#: AB086385), CDK4 (SEQ ID NO:23) was inserted into the pCX4bsr vector (GenBank#: AB086384), and dominant-negative TP53 (SEQ ID NO:25) was inserted into the pCX4.1hisD vector (GenBank#: AB086389). In addition, for the inducible c-Myc gene, an inducible expression vector was prepared by inserting the c-Myc gene into a pT-REx inducible expression vector (Invitrogen) the expression of which can be controlled with tetracycline. Viruses were produced by transferring the above retroviral expression vectors, together with pGP and pE-eco plasmids (Takara Bio; Shiga, Japan), into 293T cells (ATCC; Manassas, USA). Next, HSAE cells that had been made to express Ecotropic receptor (Eco VR) were infected with a retrovirus. The cells infected with the virus vector were cultured for two weeks in the presence of blasticidin S, G418, puromycin, zeocine and L-Histidinol, thereby carrying out selection. Regardless of which of these drugs selection was carried out with, the cultured cells were selected from a polyclonal proliferative population of infected cells.

### Cell Culture

Normal human small airway epithelial cells from a 19-year old Caucasian female were purchased from Lonza (Walkersville, Md., USA). These cells were placed on collagen-coated tissue and grown in a serum-free SAGM medium to which various growth factors supplied by Lonza had been added (SAGM Bullet Kit; Lonza). The cells were maintained in a low-oxygen environment (3% O₂ and 5% CO₂) within a wet incubator at 37° C.

### Xenograft Growth Experiment

A xenograft growth experiment was carried out on mice. Single-cell suspensions containing 1×10⁶ HSAE/4T53 cells or HSAE cells transduced with various cancer-associated genes were suspended in 50% MATRIGELT™ (BD Bioscience; San Jose, Calif., USA), and subcutaneously injected into the flanks of 6- or 7-week female athymic nude mice (BALB/c nu/nu; Japan SLC; Hamamatsu, Japan) or NOD-SCID mice (Charles River). The dimensions of the tumors were measured with a vernier caliper and the tumor volume was calculated from the following equation, based on which the tumorigenicity was estimated.

Tumor volume=ab²/2 (where a is the width, and b is the length)

Alternatively, the ratios in which measurable tumor masses formed at the sites to which the various cancer-associated gene-transduced HSAE/4T53 cells or HSAE cells had been implanted were calculated as the tumor incidence, based on which the tumorigenicity was estimated.

### Immunological Analysis and Immunohistochemical Staining

The xenograft was formalin-fixed and paraffin-embedded, after which it was sectioned and hematoxylin eosin (H&E) staining was carried out according to the normal protocol. Immunohistochemical staining was carried out in accordance with the above literature (Sasai, K., et al., Am. J. Surg. Pathol. 32, 1220-1227 (2008)) using the following antibodies: multi-cytokeratins (AE1/AE3; Dako; Glostrup, Denmark), p63 (4A4; Dako), TTF-1 (8G7G3/1; Dako), p53 (D07; Novocastra), and VIMENTIN (V9; Nichirei; Tokyo, Japan). The procedure used to carry out immunohistochemical staining was as follow. Tissue sections having a thickness of 4 μm were deparaffinized with xylene, then dehydrated with ethanol. The antigen was activated by 2 minutes of heating in 10 mM citrate buffer (pH 6.0) within a pressure cooker. The tissue slices were rehydrated with 0.01% Tween 20-containing phosphate-buffered saline (PBST) and incubated with 0.3% hydrogen peroxide, thereby deactivating the endogenous peroxidase. The tissue sections were incubated overnight at 4° C. in primary antibody at a suitable dilution concentration, then washed with PBST, following which they were incubated for 30 minutes at room temperature in Envision Dual Link solution (Dako; Glostrup, Denmark). Next, the sections were treated with diaminobenzidine (Dako), thereby visualizing the antigen-antibody reaction sites, and nucleus staining was carried out by 90 seconds of hematoxylin treatment. Slides of the tissue sections were mounted using Entellan Neu reagent (Merck; Whitehouse Station, N.J.), then sealed with a cover glass and furnished for observation.

Alcian Blue staining was carried out by formalin-fixing, paraffin-encapsulating and sectioning the xenograft, then staining the sections as described in the above literature (Steedman, H. F., Quarterly Journal of Microscopic Science, Vol. 91, p477-479 (1950)).

### Immunoblotting Technique

Protein measurement, SDS-PAGE and immunoblotting were carried out as described in the above literature (Akagi, T., et al., Mol. Cell. Biol. 22, 7015-7023 (2002)). Immunopositive protein signals were visualized by chemiluminescence using SuperSignal WestFemto reagent (Pierce, Rockford; IL, USA). Anti-c-Myc monoclonal antibodies were purchased from Invitrogen (Carlsbad, Calif., USA).

### Cloning by Limiting Dilution Technique

The preparation of cell clones from single cells was carried out as described in the above literature (Quintana, E., et al., Nature 456 (7222): 593-8 (Dec. 4, 2008)).

### Experimental Results

[1] Production of Cancer Cells Transduced with v-Src Gene and c-Myc Gene

4T53MS cells produced by transferring the c-Myc gene and the v-Src gene to HSAE/4T53 cells were subcutaneously transplanted (1×10⁶ cells/animal) in athymic nude mice (BALB/c nu/nu; Japan SLC; Hamamatsu, Japan). The xenograft growth experiment, histological analysis and immunohistochemical staining were carried out on the tumor tissue that formed. Tumor mass formation was observed in the mice into which 4T53MS cells were transplanted (FIG. 1A). The 4T53MS cells had a very high tumorigenicity. Even compared with NCI-H460 lung cancer cells, which are known to have a high tumorigenicity, the 4T53MS cells had a tumorigenicity that was about twice as high (FIG. 1B). This tumor mass was removed, tissue sections were prepared and hematoxylin-eosin (H&E) staining was carried out, whereupon cancer tissue images having a very low degree of differentiation and exhibiting no differentiated morphology were observed. The immunostaining results were negative for staining with p63, a lung epithelium marker, and were negative as well for staining with cytokeratin (antibody clone name, AE1/AE3), which is an epithelial cell marker. On the other hand, the tumor cells were strongly positive for the mesenchymal cell marker VIMENTIN. It was concluded from the above that tumors from 4T53MS cells exhibit the nature of undifferentiated cancer (FIG. 1C). In cells obtained by the gene transfer of v-Src alone or c-Myc alone to HSAE/4T53 cells (the resulting cells are referred to here as, respectively, “4T53S” and “4T53M” cells), tumorigenicity was not observed.

The 4T53MS cells had a very strong tumorigenicity. Even when subcutaneously transplanted in amounts of 1×10⁶, 1×10⁴, 1×10³ or 1×10² cells, tumor formation in NOD-SCID mice was observed at 100% probability (FIG. 2A). Even with the implantation of only ten cells, tumor formation at a probability of 50% was found to be possible (FIGS. 2A, 2B). Therefore, because 4T53MS cells are characterized by a very high tumorigenicity and differentiate into more than one type of cells, this demonstrated that 4T53MS cells have the characteristics of cancer stem cells.

4T53 mS cells produced by transferring the inducible c-Myc gene and the v-Src gene to HSAE/4T53 cells were doxycycline (DOX) treated, thereby inducing c-Myc expression in an in vitro experimental system (FIG. 3A). 4T53 mS cells cultured in the presence of DOX were transplanted subcutaneously in nude mice and DOX administration was continued. When tumor formation was observed, DOX administration was stopped (FIG. 3B). The tumor tissue obtained was stained with cytokeratin, thereby clearly distinguishing between strongly positive tumor cells and negative stromal cells. However, the characteristic images seen in, for example, adenocarcinoma or squamous cell cancer, were not observed, indicating that this was a poorly differentiated large-cell cancer-like tumor (FIG. 3C).

### [2] Production of Cancer Cells by Transfer of Human Genes

4T53RM cells obtained by the transfer of both the c-Myc gene and the KRAS^{V12} gene were subcutaneously implanted in athymic nude mice (BALB/c nu/nu; Japan SLC; Hamamatsu, Japan), and the xenograft growth experiment, histological analysis and immunohistochemical staining were carried out on the tumor tissue that formed. The 4T53RM cells had tumorigenicity. However, the tumor incidence was 38% (FIG. 4). 4T53RMB cells obtained by transferring the BCL2 gene into these 4T53RM cells were subcutaneously implanted in athymic nude mice (BALB/c nu/nu; Japan SLC; Hamamatsu, Japan), and the xenograft growth experiment, histological analysis and immunohistochemical staining were carried out on the tumor tissue that formed. The 4T53RMB cells formed tumors in nude mice at a high incidence of 80%.

In immunological examination, histologies that do not exhibit the characteristics of squamous cell cancer or adenocarcinoma were observed in both tumors from 4T53RM cells and tumors from 4T53RMB cells. These tumors were immunostained, as a result of which only a weakly positive reaction to cytokeratin was exhibited (AE1/AE3: FIG. 5). However, because a strongly positive reaction to VIMENTIN was exhibited, this was judged to be undifferentiated cancer.

4T53R cells were produced by transferring the KRAS^{V12} gene to HSAE/4T53 cells. One of the genes PIK3CA^{H1047R}, Cyclin-D1 or LKB1^{D194A} was transferred to the 4T53R cells, thereby establishing, respectively, 4T53RP cells, 4T53RD cells and 4T53RL cells. The 4T53R, 4T53RP, 4T53RD and 4T53RL cells were subcutaneously implanted in athymic nude mice (BALB/c nu/nu; Japan SLC; Hamamatsu, Japan), and the xenograft growth experiment, histological analysis and immunohistochemical staining were carried out on the tumor tissue that formed. Although tumorigenicity was not seen in the 4T53R cells, high tumor incidences were observed in the 4T53RP cells, 4T53RD cells and 4T53RL cells (these were respectively 100%, 100%, 83%; FIG. 4).

Histological analyses were carried out on tumors (xenografts) from the 4T53RP cells, 4T53RD cells or 4T53RL cells, whereupon these were found to exhibit histologies similar to actual human lung cancer tissue. In tumors formed with 4T53RP cells, histologies containing both adenocarcinoma-like regions and squamous cell cancer-like regions were observed (FIG. 6; H&E). In tumors (xenografts) formed with 4T53RD cells and 4T53RL cells, because glandular structure formation was observed and tumor stroma were abundantly present, this was regarded to have a morphology similar to that of human lung adenocarcinoma (FIG. 6; H&E). These tumors were composed of cells that are strongly positive for the epithelial cell marker cytokeratin (FIG. 6; AE1/AE3), and are also positive for lung epithelial cell marker p63 staining (FIG. 6; p63). The expression of these markers indicates that the tumors from the 4T53RP, 4T53RD and 4T53RL cells are differentiated tumors resembling human lung adenocarcinoma. Because tumors from 4T53RP cells also included squamous cell cancer-like regions, this was concluded to be a mixture of human adenocarcinoma and squamous cell carcinoma (adenosquamous cell carcinoma).

### [3] Production of Cells Having the Nature of Cancer Stem Cells for Lung Adenocarcinoma

Cancer tissue from 4T53RD cells produced by transferring both the KRAS^{V12} and Cyclin D1 genes to HSAE/4T53 cells form a glandular structure composed of a heterogeneous population of cells resembling lung adenocarcinoma. This suggests the possibility that 4T53RD cells have the nature of stem cells with the ability to differentiate into numerous types of cells.

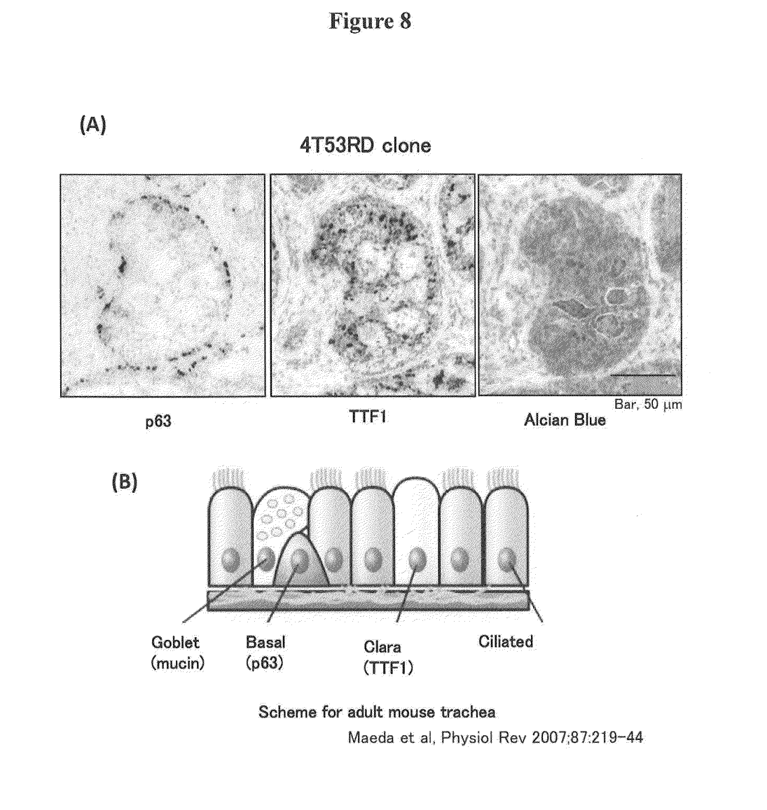

In order to confirm this possibility, 4T53RD cells were cloned by the limiting dilution method, giving several cell clones originated from a single cell. These cell clones were subcutaneously implanted in NOD-SCID mice, and the xenograft growth experiment, histological analysis and immunohistochemical staining were carried out on the tumor tissue that formed. The 4T53RD cell clones from single cells formed tumor masses and, like the parent strain of 4T53RD cells, formed tumors morphologically similar to human lung adenocarcinoma (FIG. 8A).

The mouse airway epithelium, as shown in FIG. 8B, is composed of several cell populations, including basal cells which express p63, clara cells which express TTF1, goblet cells which produce mucin, and ciliated cells.

The tumor tissue that formed upon the implantation of single cell derived 4T53RD cell clones were immunohistochemically stained, as a result of which p63 positive basal cells were present in border areas in contact with the mouse substrate. However, the portion which forms a glandular structure on the inside thereof was p63 negative, and cells positive for the TTF1 which is expressed in clara cells, and mucin-producing cells that are stained by Alcian Blue were present (FIG. 8A). Taking into consideration the fact that, according to Rock, J. R., et al. Proc. Natl. Acad. Sci. USA 106 (31), 12771-5 (Aug. 4, 2009), p63 positive basal cells have recently been shown to function as stem cells in the bronchial tubes, these results suggest that, in tumors obtained by single cell derived 4T53RD cell clones, as p63 positive cells proliferate toward the inside, they differentiate into clara cells and mucin-secreting goblet cells, thereby forming cancer tissue composed of a heterogeneous population of cells.

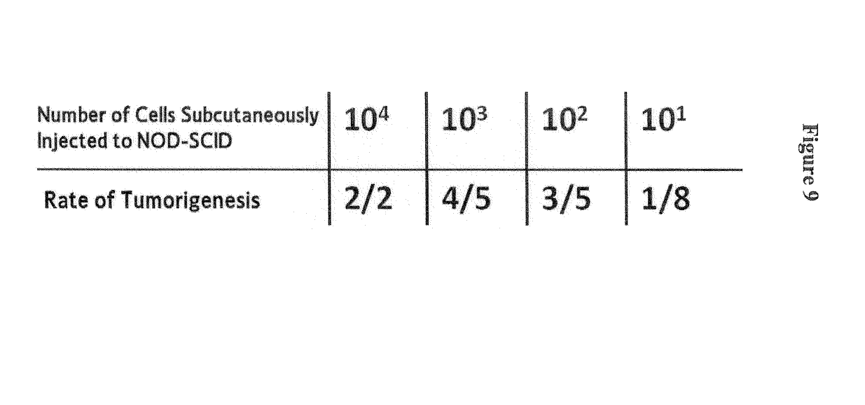

Also, 4T53RD cell clones have a strong tumorigenicity. When 1×10⁴, 1×10³, 1×10², and 1×10¹ of these cells were subcutaneously implanted, tumor formation in NOD-SCID mice at probabilities of, respectively, 100%, 80%, 60% and 13% was observed (FIG. 9).

From the above results, 4T53RD cells were shown to possess the following two properties of cancer stem cells: (1) they have the ability to differentiate from a single type of cell into a plurality of types of cells in the course of forming a tumor in vivo; and (2) even a small number of cells are capable of forming a tumor.

### [4] Production of Cells Having the Characteristic of Lung Squamous Cell Cancer

4T53RDΔNp63 cells produced by transferring the KRAS^{V12}, Cyclin D1 and TP63^{ΔN} genes to HSAE/4T53 cells were subcutaneously implanted in athymic nude mice (BALB/c nu/nu; Japan SLC; Hamamatsu, Japan), and histological analysis and immunohistochemical staining were carried out on the tumor tissue that formed.

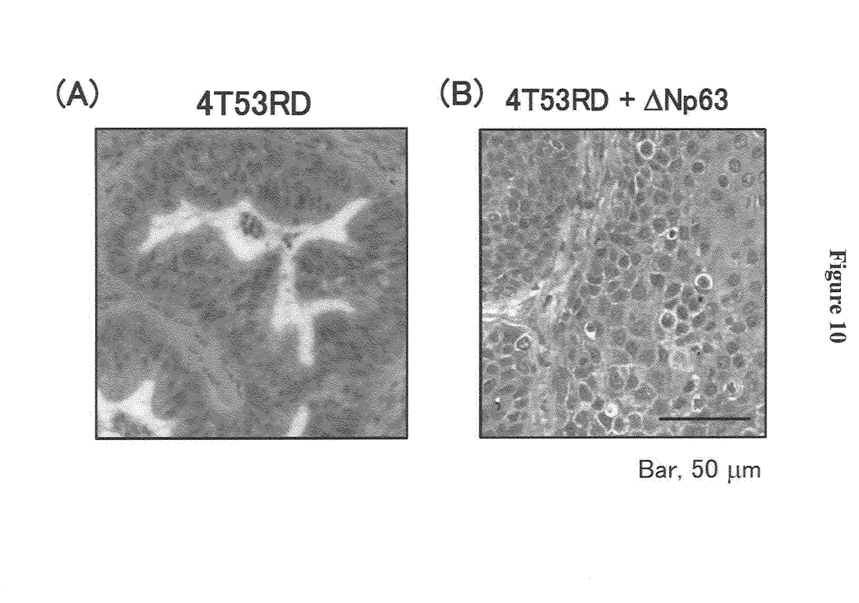

4T53RD cells produced by transferring both the KRAS^{V12} gene and the Cyclin D1 gene to HSAE/4T53 cells formed tumors having a morphology similar to that of human lung adenocarcinoma (FIG. 10A), whereas cells produced by transferring not only the KRAS^{V12} and Cyclin D1 genes but also the TP63^{ΔN} gene formed, according to histological analysis, squamous cell carcinoma-like tumor tissue (FIG. 10B).

### [5] Production of Differentiated Lung Cancer Cells Using EGFR Mutated Gene and Cyclin D1

4T53 ED cells produced by transferring both the EGFR^{T790M L858R} gene and the Cyclin D1 gene into HSAE/4T53 cells were subcutaneously implanted in athymic nude mice (BALB/c nu/nu; Japan SLC; Hamamatsu, Japan), and the xenograft growth experiment, histological analysis and immunohistochemical staining were carried out on the tumor tissue that formed.

The 4T53 ED cells exhibited 100% tumor incidence (FIG. 4), and thus were found to have a high tumorigenicity. Also, tumor tissue from 4T53 ED cells formed a particular type of differentiated tumor tissue having a morphology composed primarily of squamous epithelial-like cells among which were interspersed adenocarcinoma-like cells (FIGS. 11A to 11C).

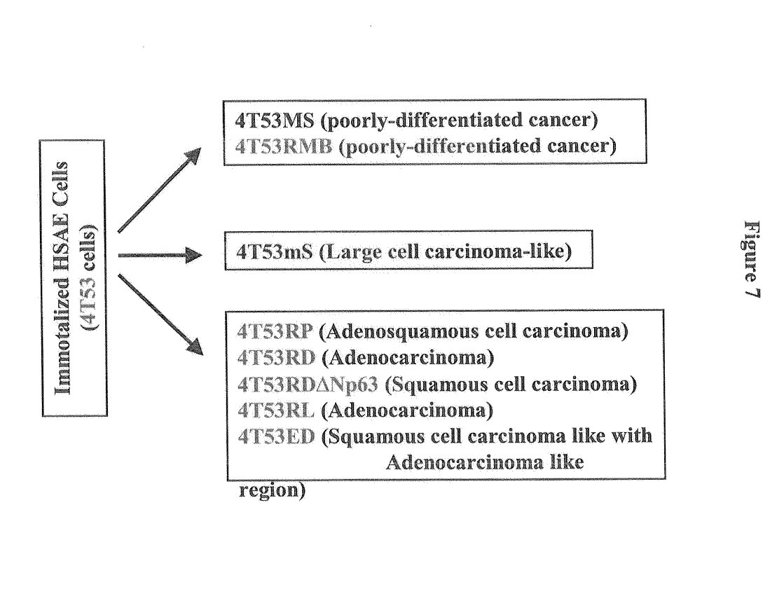

As demonstrated by the above results, by transferring cancer-associated genes into immortalized human small airway epithelial cells (HSAE/4T53 cells), it is possible to regulate the degree of tumor cell differentiation. That is, as shown in FIG. 7, it is possible to produce models of undifferentiated cancer by gene transfer involving the combination of a c-Myc gene and a v-Src gene (4T53MS cells) or the combination of a c-Myc gene, a KRAS mutated gene and a BCL2 gene (4T53RMB cells). Also, by transferring genes in the combination of an inducible c-Myc gene and a v-Src gene (4T53 mS cells), it is possible to establish a poorly differentiated lung cancer model resembling human lung large-cell cancer upon c-Myc induction. In addition, by gene transfer in any one of the following combinations, that is, the combination of a KRAS mutated gene and a PIK3CA mutated gene (4T53RP cells), the combination of a KRAS mutated gene and a Cyclin D1 gene (4T53RD cells), the combination of a KRAS mutated gene and a LKB1 mutated gene (4T53RL cells), the combination of a KRAS mutated gene, a Cyclin D1 gene and a TP63 gene (4T53RDANp63 cells), or the combination of a EGFR mutated gene and a Cyclin D1 gene (4T53ED cells), it is possible to produce human lung cancer models resembling adenocarcinoma or squamous cell carcinoma (or a mixture thereof).

### INDUSTRIAL APPLICABILITY

The present invention provides tumor cells which exhibit pathological characteristics similar to those of human lung cancer cells. Such tumor cells are useful in research on the biological mechanisms of lung cancer, the identification of target molecules for lung cancer treatment, and the screening and testing of cancer drugs. In particular, tumor cells produced by the transfer of only cancer-associated human genes faithfully recapitulate the properties of the cell, and are expected to be highly useful in the development of cancer drugs.

1. A method for producing a tumor cell by transferring one or more cancer-associated genes into an immortalized small airway epithelial cell, wherein
the immortalized small airway epithelial cell is produced by subjecting a normal small airway epithelial cell to the following treatments (1) to (3):
(1) forced expression of telomere reverse transcriptase gene;
(2) forced expression of cyclin-dependent kinase 4 gene; and
(3) induction of p53 loss of function, and wherein
the one or more cancer-associated genes comprise one or more genes selected from the group consisting of c-Myc gene, v-Src gene, KRAS mutated gene, BCL2 gene, PIK3CA mutated gene, Cyclin D1 gene, LKB1 mutated gene, TP63 gene and EGFR mutated gene.

2. The method according to claim 1, wherein the one or more cancer-associated genes comprise a combination of c-Myc gene and v-Src gene.

3. The method according to claim 1, wherein the one or more cancer-associated genes comprise a combination of c-Myc gene, KRAS mutated gene and BCL2 gene.

4. The method according to claim 1, wherein the tumor cell is an undifferentiated cancer cell.

5. The method according to claim 4, wherein the tumor cell is a cancer stem cell.

6. The method according to claim 1, wherein the one or more cancer-associated genes comprise a c-Myc gene and a v-Src gene,
and wherein the c-Myc gene is inductively expressed in the immortalized human small airway epithelial cells.

7. The method according to claim 6, wherein the tumor is a poorly differentiated lung cancer cell.

8. The method according to claim 1, wherein the one or more cancer-associated genes comprise a combination of KRAS mutated gene and a gene selected from the group consisting of PIK3CA mutated gene, Cyclin D1 gene and LKB1 mutated gene.

9. The method according to claim 8, wherein the one or more cancer-associated genes comprise a combination of KRAS mutated gene and Cyclin D1 gene.

10. The method according to claim 8, wherein the one or more cancer-associated genes comprise a combination of KRAS mutated gene, Cyclin D1 gene and TP63 gene.

11. The method according to claim 8, wherein the one or more cancer-associated genes comprise a combination of KRAS mutated gene and PIK3CA mutated gene.

12. The method according to claim 8, wherein the one or more cancer-associated genes comprise a combination of KRAS mutated gene and LKB1 mutated gene.

13. The method according to claim 1, wherein the one or more cancer-associated genes comprise a combination of EGFR mutated gene and Cyclin D1 gene.

14. The method according to claim 8, wherein the tumor cells are differentiated lung cancer cells.

15. The method according to claim 9, wherein the tumor cell is a cancer stem cell of a differentiated lung cancer.

16. The method according to claim 8, wherein the immortalized small airway epithelial cell is a mammalian cell.

17. A tumor cell produced by the method according to claim 1.

18. A tumor-bearing animal implanted with the tumor cell according to claim 17.

19. A method for screening a cancer drug, the method comprising:
(a) contacting the tumor cell of claim 17 with a candidate substance; and
(b) detecting tumor cell growth inhibiting effects.

20. The method according to claim 16, wherein the immortalized small airway epithelial cell is a primate cell.
